(19) United States
(12) Patent Application Publication
(10) Pub. No.: US 2019/0218603 A1
(43) Pub. Date: Jul. 18, 2019

(54) MICRORNA EXPRESSION SIGNATURES FOR DOUBLECORTIN-LIKE KINASE 1 (DCLK1) ACTIVITY

(71) Applicant: THE BOARD OF REGENTS OF THE UNIVERSITY OF OKLAHOMA, Norman, OK (US)

(72) Inventors: Courtney W. HOUCHEN, Edmond, OK (US); Nathaniel WEYGANT, Mustang, OK (US); Dongfeng QU, Edmond, OK (US); Randal MAY, Oklahoma City, OK (US); Parthasarathy CHANDRAKESAN, Edmond, OK (US)

(73) Assignee: The Board of Regents of the University of Oklahoma, Norman, OK (US)

(21) Appl. No.: 16/301,982

(22) PCT Filed: May 18, 2017
(86) PCT No.: PCT/US2017/033335

§ 371 Date: Nov. 15, 2018

Related U.S. Application Data
(60) Provisional application No. 62/338,732, filed on May 19, 2016.

(51) Int. Cl. C12Q 1/6837; C12Q 1/6886; C12Q 1/6825; C12Q 1/6832; C12Q 1/6806; C12Q 1/686; C12Q 1/6853

(52) U.S. Cl. CPC C12Q 1/6837; C12Q 1/6886; C12Q 1/6825; C12Q 1/6832; C12Q 2600/16; C12Q 1/686; C12Q 1/6853; C12Q 2600/158; C12Q 2600/178; C12Q 1/6806

(57) ABSTRACT

Primer and/or probe sets and arrays for determining microRNA (miRNA) expression signatures that are specific to doublecortin-like kinase 1 (DCLK1) activity, and methods of their use, are disclosed.

### CROSS REFERENCE TO RELATED APPLICATIONS/INCORPORATION BY REFERENCE STATEMENT

The present patent application claims priority to U.S. Provisional Patent Application Ser. No. 62/338,732, filed on May 19, 2016, the entire contents of which is hereby expressly incorporated herein by reference.

### BACKGROUND

Gastrointestinal cancers are commonly observed malignancies, and virtually all of these arise from normal tissue containing doublecortin-like kinase 1 (DCLK1)+tuft cells. These cells are thought to be involved in sensory functions and signaling during cellular homeostasis and in response to injury. Moreover, strongly increased expression of DCLK1 is observed in both pre-cancerous lesions and cancers of the gastrointestinal organs, suggesting clonal expansion of DCLK1+ cells during tumor initiation and/or activation of downstream oncogenic signaling. Recently, the presence of DCLK1 tumor stem and stem-like cells has been confirmed in models of colon and pancreatic cancer, elevating the importance of this marker. Moreover, the development of prognostic biomarkers in GI cancers has been slow, but developing markers based on an essential target like DCLK1 may have the potential to improve treatment strategies and increase patient quality of life and survival.

Studies in murine models show that DCLK1 both specifically identifies tumor stem and stem-like cells and can serve as a potential therapeutic anti-tumor target with no apparent toxicity to normal cells or cellular homeostasis. Moreover, DCLK1 has been tightly linked to epithelial-mesenchymal transition (EMT), which is important in the metastatic processes of many tumors including those of the gastrointestinal tract. However, although DCLK1 marks cells that initiate tumors and is expressed in the primary tumor, circulating tumor cells and in metastases, expression levels of DCLK1 may be unstable. DCLK1 expression levels have a tendency to decrease with advancing disease status possibly due to increased proliferation of tumor stem cell-derived progeny that make up the bulk of the tumor. DCLK1 expression has been investigated as a potential biomarker in colon, stomach, and pancreatic cancers.

As noted, the utility of DCLK1 expression as a biomarker may be limited because of signal instability and dilution as diverse tumor lineages proliferate within the tumor and obscure the direct measurement of DCLK1. The limitations to using DCLK1 directly as a biomarker could be overcome by developing a stable molecular signature indicative of DCLK1 activity.

MicroRNAs (miRNAs) are a uniquely stable set of ubiquitously expressed small non-coding RNAs that regulate complex processes during both homeostasis and in disease. In cancer, miRNAs modulate stemness, EMT, expression of tumor suppressor genes and oncogenes, and many other essential pathways that phenotypically affect cancer cells such as drug-resistance, tumor growth, invasion, and metastasis. MiRNAs are highly stable molecules that can be measured in biological samples including formalin-fixed paraffin embedded tissue, blood, and urine. The Cancer Genome Atlas (TCGA) Project has collected and disseminated large multi-site datasets that allow for assessment of the prognostic and diagnostic value of protein, RNA, and other markers, including miRNAs, in the setting of malignancy. Identification of a stable, surrogate miRNA-signature for DCLK1 activity in tumors would be useful, for example, to determine the nature of such a signature in patients with cancers of the colon, pancreas, and stomach.

### BRIEF DESCRIPTION OF THE DRAWINGS

Several embodiments of the present disclosure are hereby illustrated in the appended drawings. It is to be noted however, that the appended drawings only illustrate several embodiments and are therefore not intended to be considered limiting of the scope of the present disclosure.

FIG. 1A shows that DCLK1 is strongly correlated to epithelial-mesenchymal transition in all TCGA gastrointestinal cancer datasets, especially those originating in organs with tuft cells present in normal tissue.

FIG. 1B is a Circos schematic of expression of DCLK1-activity correlated miRNAs with chromosomal location. From inner to outer concentric circles: colon adenocarcinoma (COAD), esophageal carcinoma (ESCA), pancreatic adenocarcinoma (PAAD), rectal adenocarcinoma (READ), and stomach adenocarcinoma (STAD) miRNA expression correlations; representation of HG18 chromosome cytobands; combined miRNA expression correlations for all 5 cancers; consensus significant miRNA expression correlations with labels.

FIG. 2A is a heatmap demonstrating dysregulated expression of the 15-miRNA signature between DCLK1-low and DCLK1-high colon tumor patients from the TCGA COAD dataset.

FIG. 2B is a heatmap demonstrating dysregulated expression of the 15-miRNA signature between DCLK1-low and DCLK1-high rectal tumor patients from the TCGA READ dataset.

FIG. 2C is a heatmap demonstrating dysregulated expression of the 15-miRNA signature between DCLK1-low and DCLK1-high pancreas tumor patients from the TCGA PAAD dataset.

FIG. 2D is a heatmap demonstrating dysregulated expression of the 15-miRNA signature between DCLK1-low and DCLK1-high esophagus tumor patients from the TCGA ESCA dataset.

FIG. 2E is a heatmap demonstrating dysregulated expression of the 15-miRNA signature between DCLK1-low and DCLK1-high stomach tumor patients from the TCGA STAD dataset.

FIG. 3A shows boxplots demonstrating increased EMT status (left panel) and increased expression of DCLK1 (right panel) between miRNA-signature low (miR Low) and miRNA-signature high (miR high) tumors in all 5 tuft cell-containing GI cancers from the TCGA COAD, ESCA, PAAD, READ, and STAD datasets (p<0.0001 for all comparisons).

FIG. 3B shows that SW480 cells express high levels of DCLK1. Downregulation of DCLK1 in this cell line via DCLK1-targeted siRNA results in upregulation of miR-141, miR-200a-b, miR-425, and miR-532 (*p<0.05).

FIG. 3C shows that overexpression of DCLK1 in the AsPC-1 cell line which expresses nearly undetectable levels of DCLK1 results in downregulation of miR-141, miR-200a-b, miR-425, and miR-532 (*p<0.05).

FIG. 4A is a heatmap of significant pathways induced by the miRNA-signature as determined by KEGG pathway enrichment analysis using DIANA miRPath including colorectal, pancreatic, and renal cell cancer pathways—all cancers in which DCLK1 activity has been demonstrated to have significant functional activity.

FIG. 4B is a Kaplan-Meier survival analysis demonstrating that a DCLK1-activity based 15-miRNA signature predicts overall (p<0.05) and recurrence-free survival (p<0.005) in colon cancer patients.

FIG. 4C is a Kaplan-Meier survival analysis demonstrating that a DCLK1-activity based 15-miRNA signature predicts overall (p<0.05) survival in pancreatic cancer patients.

FIG. 4D is a Kaplan-Meier survival analysis demonstrating that a DCLK1-activity based 15-miRNA signature predicts overall (p<0.05) and recurrence-free survival (p<0.01) in stomach cancer patients).

FIG. 5A shows a subgroup analysis of overall survival in low risk compared to high risk miRNA-signature tumors in TCGA COAD, PAAD, and STAD datasets demonstrating the prognostic value of the signature in certain subsets of patients.

FIG. 5B shows a Kaplan-Meier analysis of select patient subsets demonstrating the prognostic significance of the signature in Stage I-II colon cancer patients (p<0.01), pancreatic cancer patients under 65 years old (OS: p<0.02; RFS: p<0.008), and stomach cancer patients receiving radiation therapy (Log-Rank: p=0.078; Gehan-Breslow: p=0.022).

FIG. 6A shows a comparison of observed survival by miRNA signature expression among patients with definite outcomes in the TCGA colon cancer datasets.

FIG. 6B shows a comparison of observed survival by miRNA signature expression among patients with definite outcomes in the TCGA pancreatic cancer datasets.

FIG. 6C shows a comparison of observed survival by miRNA signature expression among patients with definite outcomes in the TCGA stomach cancer datasets.

FIG. 6D depicts predicted prognostic receiver operating characteristic (ROC) data for colon (COAD), pancreas (PAAD), and stomach (STAD) cancer datasets as well as relevant subgroups as modeled by the prognostic ROC statistical package and observed survival in patients with known outcomes at 18 months, 3 years, and 5 years post-diagnosis.

FIG. 7A shows a Kaplan-Meier survival analysis demonstrating that colon cancer patients with high DCLK1 gene expression (top 25^{th} percentile) have significantly reduced overall (p<0.05, HR: 2.214).

FIG. 7B shows a Kaplan-Meier survival analysis demonstrating recurrence-free (p<0.05, HR: 2.433) survival in colon cancer patients with high DCLK1 gene expression (top 25^{th} percentile) compared to those with low expression (bottom 25^{th} percentile).

FIG. 8 shows several clinical patient characteristics for the colon, pancreatic, and stomach cancer patients included in the miRNA-signature survival study. Asterisks denote statistically significant parameters associated with reduced overall survival.

FIG. 9 is a flowchart demonstrating how the DCLK1-based signature was derived from the 5 gastrointestinal cancers.

FIG. 10A shows a subgroup analysis of recurrence-free survival in low risk compared to high risk miRNA-signature tumors in TCGA COAD datasets demonstrating the prognostic value of the signature in certain subsets of patients.

FIG. 10B shows a subgroup analysis of recurrence-free survival in low risk compared to high risk miRNA-signature tumors in TCGA PAAD datasets demonstrating the prognostic value of the signature in certain subsets of patients.

FIG. 10C shows a subgroup analysis of recurrence-free survival in low risk compared to high risk miRNA-signature tumors in TCGA STAD datasets demonstrating the prognostic value of the signature in certain subsets of patients.

### DETAILED DESCRIPTION

In certain embodiments the present disclosure is directed to primer and/or probe sets for determining a microRNA (miRNA) expression signature that is specific to doublecortin-like kinase 1 (DCLK1) activity in cancer cells. Because DCLK1 is a tumor stem cell protein, as the tumor produces progeny from the tumor stem cells the ability to measure DCLK1 and other related tumor stem cell markers directly by standard techniques may be impaired. An miRNA signature allows stable measurement of tumor stem cell activity. In certain embodiments, the signature can be used to predict overall survival and/or cancer recurrence in, for example, pancreatic, colon, and stomach cancer patients. In one non-limiting embodiment, the miRNA signature is based on the expression values for 5-15 miRNAs (also shown in Table 1), including hsa-miR-99a, hsa-Let-7c, hsa-miR-125b-1, hsa-miR-125b-2, hsa-miR-218-1, hsa-miR-218-2, hsa-miR-100, which are upregulated, and hsa-miR-532, hsa-miR-200a, hsa-miR-200b, hsa-miR-429, hsa-miR-425, hsa-miR-192, hsa-miR-194-2, and hsa-miR-141, which are downregulated. The signature is constructed from expression values measured using at least one of the primers and/or probe sets for the 3′ or 5′ isoform of each of the 5-15 miRNAs.

Before further describing various embodiments of the kits, arrays, panels, compounds, compositions, and methods of the present disclosure in more detail by way of exemplary description, examples, and results, it is to be understood that the kits, arrays, panels, compounds, compositions, and methods of present disclosure are not limited in application to the details of specific embodiments and examples as set forth in the following description. The description provided herein is intended for purposes of illustration only and is not intended to be construed in a limiting sense. As such, the language used herein is intended to be given the broadest possible scope and meaning; and the embodiments and examples are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the present disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure. All of the kits, arrays, panels, compounds, compositions, and methods and application and use thereof disclosed herein can be made and executed without undue experimentation in light of the present disclosure. Thus, while the kits, arrays, panels, compounds, compositions, and methods of the present disclosure have been described in terms of particular embodiments, it will be apparent to those of skill in the art that variations may be applied to the kits, arrays, panels, compounds, compositions, and methods and in the steps or in the sequence of steps of the methods described herein without departing from the concept, spirit, and scope of the inventive concepts.

All patents, published patent applications, and non-patent publications mentioned in the specification or referenced in any portion of this application, are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular. Where used herein, the specific term “single” is limited to only “one”.

As utilized in accordance with the methods, compounds, and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment and may be included in other embodiments. The appearances of the phrase “in one embodiment” in various places in the specification are not necessarily all referring to the same embodiment and are not necessarily limited to a single or particular embodiment.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The compounds or conjugates of the present disclosure may be combined with one or more pharmaceutically-acceptable excipients, including carriers, vehicles, and diluents which may improve solubility, deliverability, dispersion, stability, and/or conformational integrity of the compounds or conjugates thereof.

By “biologically active” is meant the ability to modify the physiological system of an organism without reference to how the active agent has its physiological effects.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

Non-limiting examples of animals within the scope and meaning of this term include dogs, cats, rats, mice, guinea pigs, chinchillas, horses, goats, cattle, sheep, zoo animals, Old and New World monkeys, non-human primates, and humans.

“Treatment” refers to therapeutic treatments. “Prevention” refers to prophylactic or preventative treatment measures or reducing the onset of a condition or disease. The term “treating” refers to administering the composition to a subject for therapeutic purposes and/or for prevention.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable therapeutic or treatment effect in a subject without excessive adverse side effects (such as substantial toxicity, irritation and allergic response) commensurate with a reasonable benefit/risk ratio when used in the manner of the present disclosure. The effective amount for a subject will depend upon the subject's type, size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition, disease or symptom thereof. A detectable or measurable improvement includes a subjective or objective decrease, reduction, inhibition, suppression, limit or control in the occurrence, frequency, severity, progression, or duration of the condition or disease, or an improvement in a symptom or an underlying cause or a consequence of the disease, or a reversal of the disease. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a disease or condition, or consequences of the disease or condition in a subject.

A decrease or reduction in worsening, such as stabilizing the condition or disease, is also a successful treatment outcome. A therapeutic benefit therefore need not be complete ablation or reversal of the disease or condition, or any one, most or all adverse symptoms, complications, consequences or underlying causes associated with the disease or condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition or disease (e.g., stabilizing), over a short or long duration of time (hours, days, weeks, months, etc.). Effectiveness of a method or use, such as a treatment that provides a potential therapeutic benefit or improvement of a condition or disease, can be ascertained by various methods and testing assays.

The term “homologous” or “% identity” as used herein means a nucleic acid (or fragment thereof) or a protein (or a fragment thereof) having a degree of homology to the corresponding natural reference nucleic acid or protein that may be in excess of 70%, or in excess of 80%, or in excess of 85%, or in excess of 90%, or in excess of 91%, or in excess of 92%, or in excess of 93%, or in excess of 94%, or in excess of 95%, or in excess of 96%, or in excess of 97%, or in excess of 98%, or in excess of 99%. For example, in regard to peptides or polypeptides, the percentage of homology or identity as described herein is typically calculated as the percentage of amino acid residues found in the smaller of the two sequences which align with identical amino acid residues in the sequence being compared, when four gaps in a length of 100 amino acids may be introduced to assist in that alignment (as set forth by Dayhoff, in Atlas of Protein Sequence and Structure, Vol. 5, p. 124, National Biochemical Research Foundation, Washington, D.C. (1972)). In one embodiment, the percentage homology as described above is calculated as the percentage of the components found in the smaller of the two sequences that may also be found in the larger of the two sequences (with the introduction of gaps), with a component being defined as a sequence of four, contiguous amino acids. Also included as substantially homologous is any protein product which may be isolated by virtue of cross-reactivity with antibodies to the native protein product. Sequence identity or homology can be determined by comparing the sequences when aligned so as to maximize overlap and identity while minimizing sequence gaps. In particular, sequence identity may be determined using any of a number of mathematical algorithms. A non-limiting example of a mathematical algorithm used for comparison of two sequences is the algorithm of Karlin & Altschul, Proc. Natl. Acad. Sci. USA 1990, 87, 2264-2268, modified as in Karlin & Altschul, Proc. Natl. Acad. Sci. USA 1993, 90, 5873-5877.

In one embodiment “% identity” represents the number of amino acids or nucleotides which are identical at corresponding positions in two sequences of a protein having the same activity or encoding similar proteins. For example, two amino acid sequences each having 100 residues will have 95% identity when 95 of the amino acids at corresponding positions are the same.

Another example of a mathematical algorithm used for comparison of sequences is the algorithm of Myers & Miller, CABIOS 1988, 4, 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988, 85, 2444-2448.

Another algorithm is the WU-BLAST (Washington University BLAST) version 2.0 software (WU-BLAST version 2.0 executable programs for several UNIX platforms). This program is based on WU-BLAST version 1.4, which in turn is based on the public domain NCBI-BLAST version 1.4 (Altschul & Gish, 1996, Local alignment statistics, Doolittle ed., Methods in Enzymology 266, 460-480; Altschul et al., Journal of Molecular Biology 1990, 215, 403-410; Gish & States, Nature Genetics, 1993, 3: 266-272; Karlin & Altschul, 1993, Proc. Natl. Acad. Sci. USA 90, 5873-5877; all of which are incorporated by reference herein).

In addition to those otherwise mentioned herein, mention is made also of the programs BLAST, gapped BLAST, BLASTN, BLASTP, and PSI-BLAST, provided by the National Center for Biotechnology Information. These programs are widely used in the art for this purpose and can align homologous regions of two amino acid sequences. In all search programs in the suite, the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Specific amino acids may be referred to herein by the following designations: alanine: ala or A; arginine: arg or R; asparagine: asn or N; aspartic acid: asp or D; cysteine: cys or C; glutamic acid: glu or E; glutamine: gin or Q; glycine: gly or G; histidine: his or H; isoleucine: ile or I; leucine: leu or L; lysine: lys or K; methionine: met or M; phenylalanine: phe or F; proline: pro or P; serine: ser or S; threonine: thr or T; tryptophan: trp or W; tyrosine: tyr or Y; and valine: val or V.

The terms “polynucleotide sequence” or “nucleic acid,” as used herein, include any polynucleotide sequence which encodes a mutant peptide including polynucleotides in the form of RNA, such as mRNA, or in the form of DNA, including, for instance, cDNA and genomic DNA obtained by cloning or produced by chemical synthetic techniques or by a combination thereof. The DNA may be double-stranded or single-stranded. Single-stranded DNA may be the coding strand, also known as the sense strand, or it may be the non-coding strand, also referred to as the anti-sense strand. The polynucleotide sequence encoding a mutant peptide, or encoding a therapeutically-effective fragment of a mutant peptide can be substantially the same as the coding sequence of the endogenous coding sequence as long as it encodes a biologically active mutant peptide. Further, the mutant peptide, or therapeutically-effective fragment of a mutant peptide may be expressed using polynucleotide sequence(s) which differ in codon usage due to the degeneracies of the genetic code or allelic variations. Moreover, the mutant peptides of the present disclosure and the nucleic acids which encode them include peptide and nucleic acid variants which comprise additional conservative substitutions. For example, the peptide variants include, but are not limited to, variants that are not exactly the same as the sequences disclosed herein, but which have, in addition to the substitutions explicitly described for various sequences listed herein, conservative substitutions of amino acid residues which do substantially not impair the agonistic or antagonistic activity or properties of the variants described herein. Examples of such conservative amino acid substitutions include, but are not limited to, ala to gly, ser, or thr; arg to gln, his, or lys; asn to asp, gin, his, lys, ser, or thr; asp to asn or glu; cys to ser; gin to arg, asn, glu, his, lys, or met; glu to asp, gin, or lys; gly to pro or ala; his to arg, asn, gin, or tyr; ile to leu, met, or val; leu to ile, met, phe, or val; lys to arg, asn, gin, or glu; met to gin, ile, leu, or val; phe to leu, met, trp, or tyr; ser to ala, asn, met, or thr; thr to ala, asn, ser, or met; trp to phe or tyr; tyr to his, phe or trp; and val to ile, leu, or met.

The term “antisense” refers to a polynucleotide or oligonucleotide molecule that is substantially complementary or 100% complementary to a particular polynucleotide or oligonucleotide molecule (RNA or DNA), i.e., a “sense” strand, or portion thereof. For example, the antisense molecule may be complementary in whole or in part to a molecule of messenger RNA, miRNA, pRNA, tRNA, rRNA of hnRNA, or a sequence of DNA that is either coding or non-coding.

The term “operably linked” where used herein refers to an association of two chemical moieties linked in such a way so that the function of one is not affected by the other, e.g., an arrangement of elements wherein the components so described are configured so as to perform their usual function. The two moieties may be linked directly, or may be linked indirectly via a linker sequence of molecule.

The term “primer” refers to an oligonucleotide sequence which serves as a starting point for DNA synthesis in the polymerase chain reaction (PCR). A primer generally comprises from about 12 to about 30 nucleotides and hybridizes with a complementary region of a target sequence, for example a microRNA molecule.

The term “probe” refers to an oligonucleotide which is bound to or configured to bind to a target sequence, and includes for example, an antisense nucleic acid sequence which is designed to hybridize by a sequence-specific method with a complementary region of a specific nucleic acid sequence such as a target nucleic acid, such as an miRNA as disclosed herein. An oligonucleotide probe can comprise any number of nucleotides, such as 10 to 25, as long as the oligonucleotide probe comprises a sufficient number of nucleotides to bind to the target nucleic acid with the necessary specificity for the particular use of the probe. For purposes of quantification of the probe-target sequence complex, the probe may further optionally comprise a tag or label operably linked thereto, wherein the tag or label comprises, for example, a fluorescent (e.g., fluorophore), luminescent, or chemiluminescent label or reporter group.

The term “fluorophore” or “fluorochrome” or “fluorescent species” or “fluorescent label” or “fluorescent tag,” as used herein indicates a substance which itself fluoresces or can be made to fluoresce. Each term is interchangeable. Fluorophores can be used alone or covalently attached (“operably-linked”) or non-covalently linked to another molecule, such as an oligonucleotide primer, probe, or miRNA, such as described herein. The process of covalently attaching a fluorophore to another molecule or compound is referred to as “fluorescent labeling” and may be conducted by, for example, an enzyme effective in forming the covalent bond therebetween.

Examples of fluorophores which may be used in various embodiments of the present disclosure include but are not limited to: hydroxycoumarin, methoxycoumarin, Alexa fluor 345, aminocoumarin, 7-diethylaminocoumarin-3-carboxylic acid, Cy2 (cyanine 2), FAM, Alexa fluor 350, Alexa fluor 405, Alexa fluor 488, Fluorescein (FITC), Alexa fluor 430, Alexa fluor 532, HEX 535, Cy3, Alexa fluor 546, Alexa fluor 555, R-phycoerythrin (PE), tetramethyl rhodamine (TRITC), Rhodamine Red-X, Tamara, Cy3.5, Rox, Alexa fluor 568, Red 613 480, Texas Red 615, Alexa fluor 594, Alexa fluor 633, Allophycocyanin, Alexa fluor 647, Cy5, Alexa fluor 660, Cy5.5, TruRed 490, Alexa fluor 680, Alexa fluor 750, Cy7, DAPI, QSY 7, QSY 33, dabsyl, BODIPY FL, BODIPY630/650, BODIPY 650/665, BODIPY TMR-X, BODIPY TR-X, Hoechst 33258, SYTOX blue, Hoechst 33342, YOYO-1 509, SYTOX green, TOTO1, TO-PRO-1, SYTOX orange, Chromomycin A3, Mithramycin, propidium iodide, ethidium bromide, Pacific Orange, Pacific Green, Pacific Blue, Oregon Green 488, Oregon Green 514, red fluorescent protein (RFP), green fluorescent protein (GFP), and cyan fluorescent protein (CFP).

As noted above, in certain embodiments the present disclosure is directed to microRNA (miRNA) signatures, and/or to sets of primer sequences or probes for expressing or detecting such miRNA signatures, that are specific to doublecortin-like kinase 1 (DCLK1) activity in cancer cells. The miRNAs are obtained from a patient's (subject's) biological specimen (blood, urine, tissue, cerebrospinal fluid, etc), and the expression values thereof are obtained by real-time RT-PCR, RNA-sequencing, or other molecular biology techniques (e.g., probe hybridization), for example. An overall signature score is then calculated based on the miRNA expression levels. The score can then be used to predict patient overall and recurrence-free survival and potentially other characteristics in cancers. For example, the signature can be used to predict risk of recurrence even in early stage colon cancer patients, which could identify patients who need more aggressive therapy and monitoring resulting in increased patient lifespan. Another example is the use of the signature to predict overall survival in pancreatic, colon, stomach, rectal, esophageal, bladder, uterine, ovarian, and lung cancer patients.

The resulting score from this calculation will be compared to the scores among the standards. This will allow classification of the patient's disease into a risk group. A patient with a high score will be classified as high-risk which may indicate, for instance, that although the patient has been classified as Stage I, the expected overall and recurrence-free survival for the patient will be comparable to a patient with Stage III-IV disease. Consequently, the patient may be monitored and treated more aggressively to prolong disease-free status and survival. Conversely, a patient with a low score can be classified as low-risk which may indicate, for instance, that the although the patient has been classified as Stage I, the expected overall and recurrence-free survival for the patient will be better than the average Stage I patient. Consequently, monitoring of this patient may be reduced, allowing for the use of clinical resources on patients who are more at risk.

As noted above, microRNAs demonstrate exceptional stability even in difficult biological samples such as formalin-fixed paraffin-embedded tissue sections, blood, and urine, and other tissues and have been used as prognostic biomarkers in a number of cancers. Moreover, several recent reports have suggested that DCLK1 regulates EMT through a miRNA-dependent mechanism, which has also been confirmed in pancreatic and colon cancer using tumor xenograft models. In the present work, miRNA and RNA-sequencing datasets made available by The Cancer Genome Atlas Project were utilized to determine a stable surrogate miRNA signature for DCLK1 activity in GI cancers. The signature score may be comprised of measurements of the expression of 5, 6, 7, 8, 9, 10, 11, 12, 13, 14, 15, or more miRNAs which are correlated with DCLK1 activity. Measurements of the miRNA expression can be obtained for example using primer sequences which are specific for the miRNA molecules in a real-time PCR process, such as described in further detail below. In at least one embodiment, the signature is derived from measurements of 15 miRNAs. Furthermore, these miRNA signatures have been subjected to KEGG pathway analysis confirming their functional relevance through their association with cancer initiation and progression related pathways.

Materials and Methods

TCGA Pan-Gastrointestinal Cancer Data

The miRNA and RNA-seq datasets from February, 2015 data runs for colon adenocarcinoma (COAD), esophageal carcinoma (ESCA), liver hepatocellular carcinoma (LIHC), pancreatic adenocarcinoma (PAAD), rectal adenocarcinoma (READ), and stomach adenocarcinoma (STAD) were downloaded from the UCSC Cancer Genome Browser.

Determination of DCLK1-Associated miRNAs

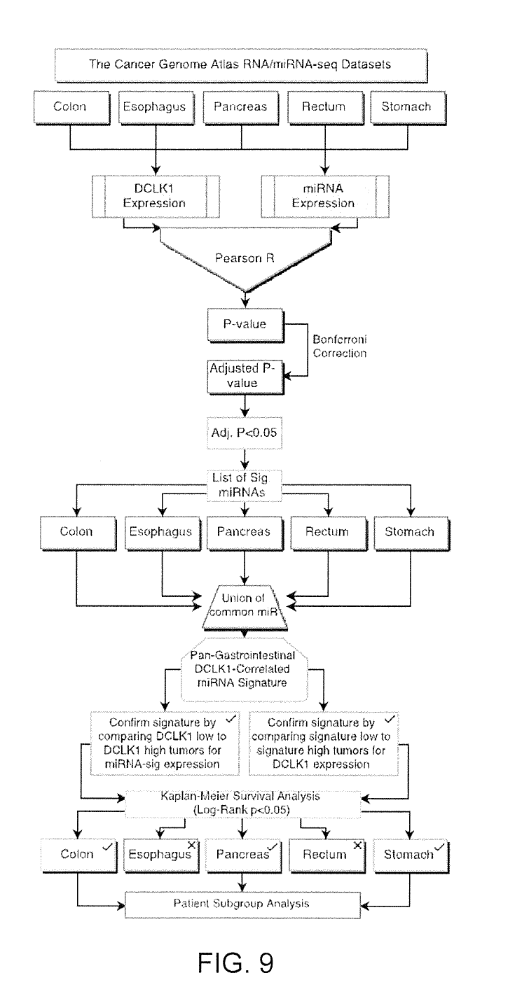

Illumina HiSeq V2 RNAseq and miRNA seq data were loaded into R v3.2 and Pearson correlations were calculated for each miRNA against DCLK1 mRNA expression in the 5 cancers derived from organs that are thought to contain tuft cells (colon, esophagus, pancreas, rectum, and stomach). The resulting correlation p-values were adjusted using the Bonferroni correction for each cancer correcting for multiple comparisons and reducing false discoveries. A Bonferroni-adjusted p-value <0.05 was considered significant. Consensus miRNAs that were significantly correlated to DCLK1 expression in all cancer types were selected to create a DCLK1 miRNA-derived signature (FIG. 9).

KEGG Pathway Analysis

Kyoto Encyclopedia of Genes and Genomes (KEGG) curated pathway analysis (pathways union) was performed using DIANA miRPath v.2.0 using Tarbase as a reference. All miRNAs with Tarbase references were included in the analysis and a targeted pathway heatmap was generated with a P-value threshold of 0.05.

Statistical Analysis

Basic statistical analyses were performed in R v3.2 and Graphpad Prism 6.0. Kaplan-Meier Survival analyses were performed in Graphpad Prism 6.0. Cox regression analyses were performed using IBM SPSS Statistics 22. Circos plots for miRNAs across cancers were generated using the RCircos R package. Correlation plots were generated using the corrplot R package. Heatmaps were generated using Genesis. Receiver operating characteristic predictions were generated using the PrognosticROC R package.

Clinical Patient Characteristics

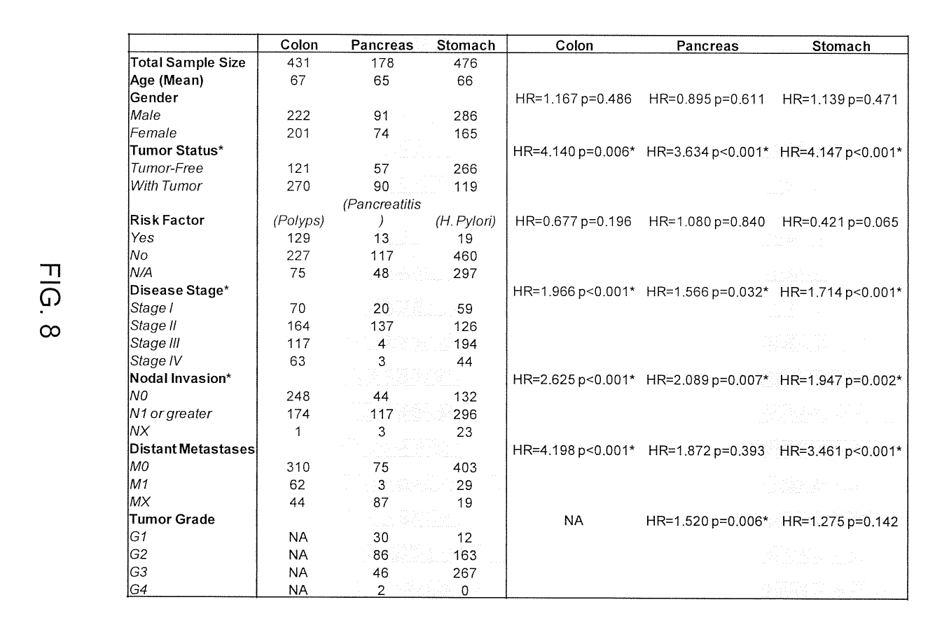

Only publicly available, de-identified data were accessed from TCGA for the analyses reported here. Basic characteristics of the patients used in the survival analyses (colon, pancreas, and stomach) are provided in FIG. 8. The average patient age was between 65 and 67 years for all three cancers. Gender was split approximately evenly between males and females for colon and pancreatic cancer, but the number of males in the stomach cancer group was significantly greater (286 males vs 165 females). Cox regression analysis demonstrated that tumor burden, disease stage, and nodal invasion were important survival factors in all three cancers while distant metastases were factors in colon and stomach cancer.

Cell Lines

SW480 colon cancer and AsPC-1 pancreatic cancer cell lines were obtained from ATCC and cultured at 37° C. in RPMI medium with 10% FBS.

Overexpression and siRNA-Mediated Knockdown of DCLK1

DCLK1 isoform 1 or vector control was expressed in AsPC-1 cells utilizing lentivirus. Overexpression was confirmed by Western blot. Knockdown of DCLK1 was achieved via transfecting SW480 cells with 50 nM of DCLK1-specific siRNA (Santa Cruz Biotechnology; SC-456178) or scrambled siRNA confirmed not to target any human genes for 72 h using Lipofectamine 3000 (Sigma). Efficient knockdown was confirmed by Western blot.

Western Blot

Western blotting was performed using specific primary antibodies against DCLK1 (Abcam 88484) and Beta-Actin (Santa Cruz Biotechnology; SC-1616), and IRdye 700 and 800 secondary antibodies (Licor). Results were visualized on a Licor Odyssey Infrared Imager and analyzed in ImageStudio (Licor).

miRNA-Specific qPCR

Total miRNAs were isolated from treated cells using a miRNeasy kit (Qiagen) according to the manufacturers instructions. Mature miRNAs were amplified by polyadenylation followed by reverse-transcription using an All-in-One miRNA First Strand cDNA Synthesis Kit (Genecopoeia). Following reverse transcription, qPCR was performed using experimentally validated, specific commercial miRNA primers (Genecopoeia). Results were calculated via the delta-delta CT method using U6 as a housekeeping miRNA.

The following natural miRNA sequences detected herein are known but are displayed in Table 1 for convenience. As noted in further detail below, the present disclosure also includes by reference antisense RNA sequences of the sequences of Table 1, as well as of subportions thereof having at least 12 nucleotides.

TABLE 1
Micro RNA 5′ and 3′ isoform sequences

| | microRNA | 5′ Sequence | SEQ ID | 3′ Sequence | SEQ ID |
|---|---|---|---|---|---|
| 1. | hsa-miR-99a | aacccguagauccgaucuugug | 1 | caagcucgcuucuaugggucug | 2 |
| 2. | hsa-miR-100 | aacccguagauccgaacuugug | 3 | caagcuuguaucuauagguaug | 4 |
| 3. | hsa-miR-125b-1 | ucccugagacccuaacuuguga | 5 | acggguuaggcucuugggagcu | 6 |
| 4. | hsa-miR-125b-2 | ucccugagacccuaacuuguga | 7 | ucacaagucaggcucuunggac | 8 |
| 5. | hsa-miR-141 | caucuuccaguacaguguugga | 9 | uaacacugucugguaaagaugg | 10 |
| 6. | hsa-miR-192 | cugaccuaugaauugacagcc | 11 | cugccaauuccauaggucacag | 12 |
| 7. | hsa-miR-194-2 | uguaacagcaacuccaugugga | 13 | ccaguggggcugcuguuaucug | 14 |
| 8. | hsa-miR-200a | caucuuaccggacagugcuuga | 15 | uaacacugucugguaacgaugu | 16 |
| 9. | hsa-miR-200b | caucuuacugggcagcauugga | 17 | uaauacugccugguaaugauga | 18 |
| 10. | hsa-miR-218-1 | uugugcuugaucuaaccaugu | 19 | augguuccaucaagcaccaugg | 20 |
| 11. | hsa-miR-218-2 | uugugcuugaucuaaccaugu | 21 | caugguucugucaagcaccgcg | 22 |
| 12. | hsa-miR-425 | aaugacacgaucacucccguuga | 23 | aucgggaaugucguguccgccc | 24 |
| 13. | hsa-miR-429 | | | uaauacugucugguaaaaccgu | 25 |
| 14. | hsa-miR-532 | caugccuugaguguaggaccgu | 26 | ccucccacacccaaggcuugca | 27 |
| 15. | hsa-miR-Let7c | uaagguaguagguuguaugguu | 28 | cuguacaaccuucuagcuuucc | 29 |

Results

DCLK1 Expression is Correlated to EMT Across Gastrointestinal Cancers

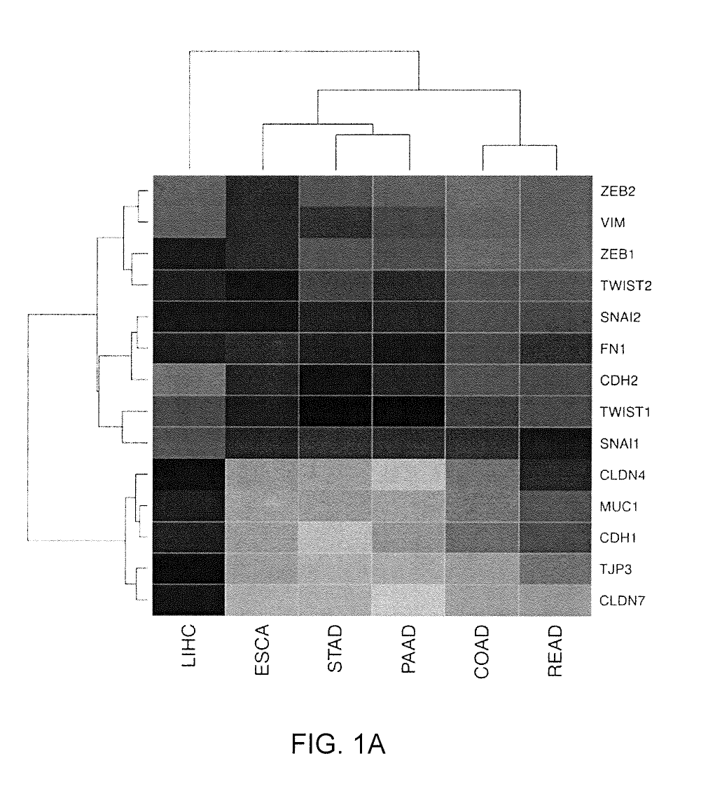

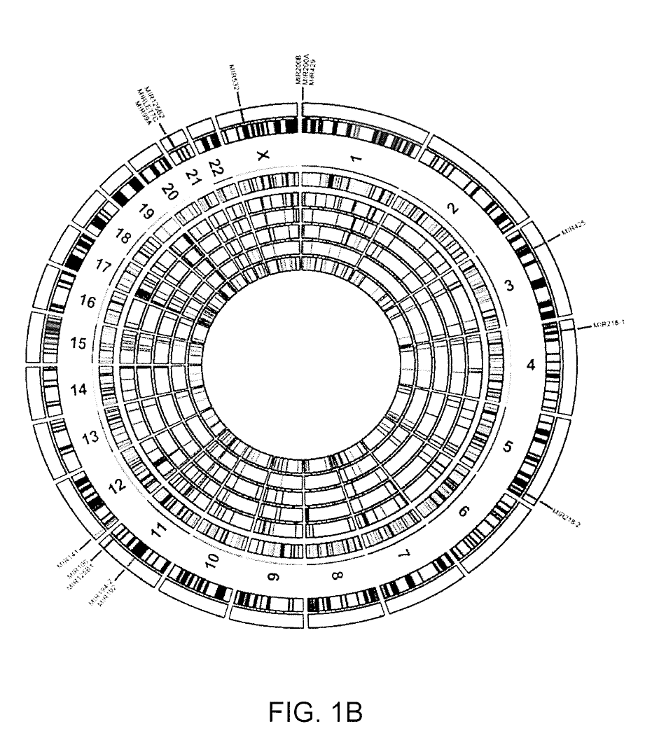

Analysis of all 6 cancer types demonstrated a strong correlation between DCLK1 mRNA expression and epithelial-mesenchymal transition as determined by the EMT spectrum score previously described (FIG. 1A). DCLK1 was most strongly correlated to EMT in colon and rectal cancers followed by cancers of the pancreas, stomach, esophagus, and liver. Although DCLK1 was significantly correlated to EMT in liver cancer, the level of correlation was approximately 3-fold less when compared to colon and almost 2-fold less than the next least correlated cancer (FIG. 1A). It appears that in hepatocellular cancer, EMT transcription factors and mesenchymal markers are correlated with DCLK1, but the loss of epithelial marker expression is not. This finding suggests that EMT in GI-tract cancers may be a process that is directly related to the presence of tuft cells that are known to be present in the esophagus, stomach, intestine, and pancreas but not the liver.

Determination of a microRNA Signature for DCLK1 Tumor Activity

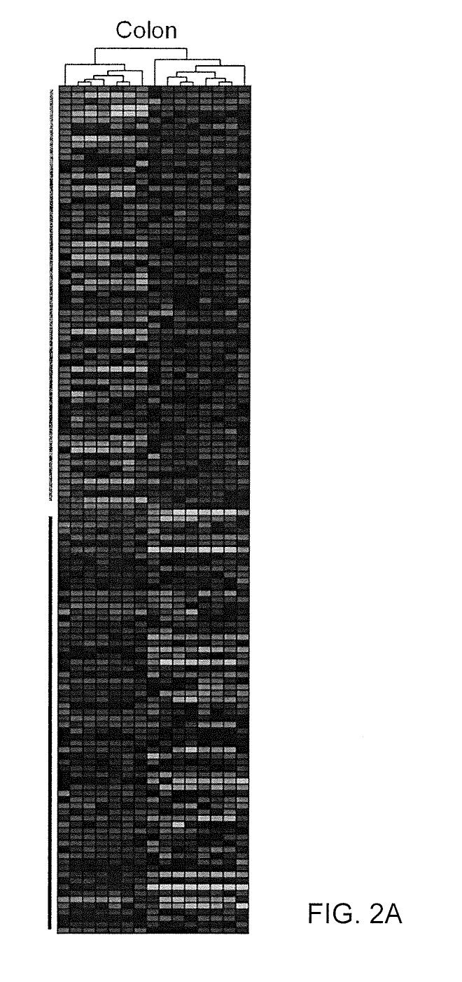

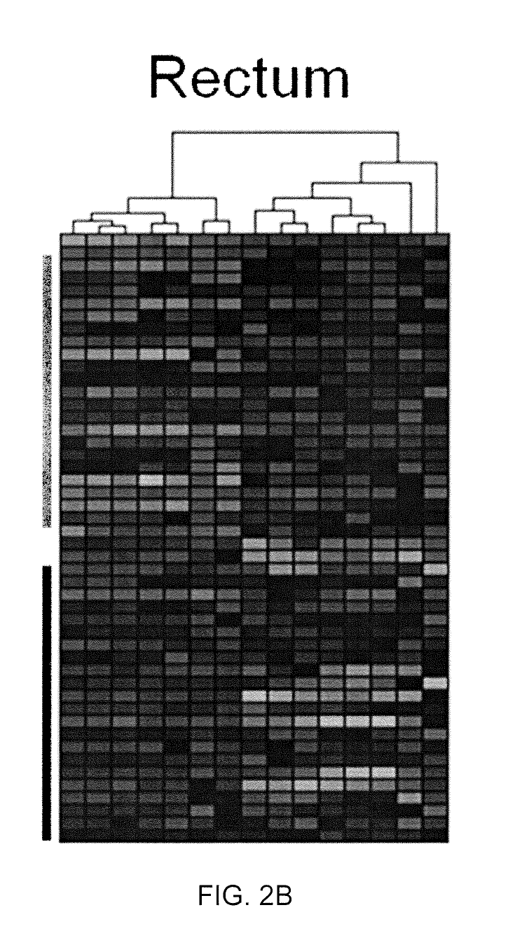

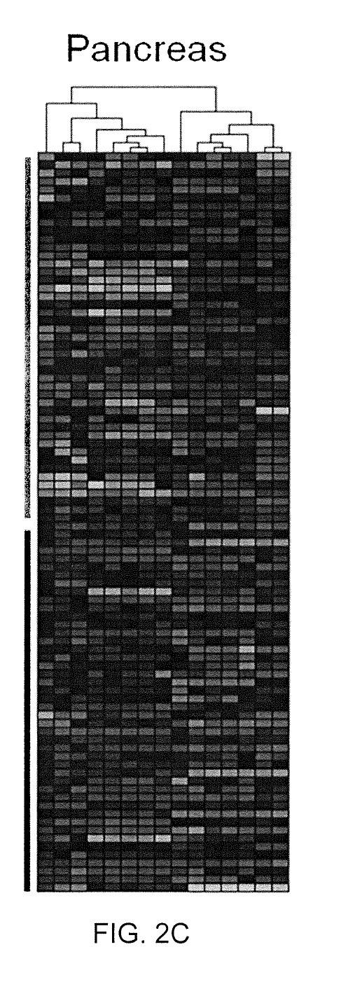

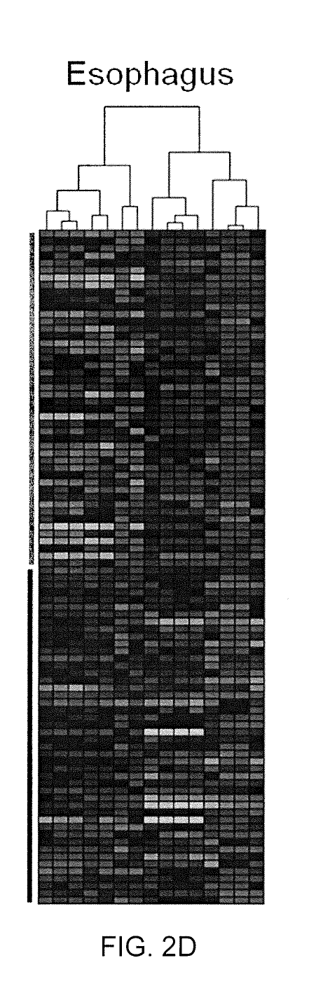

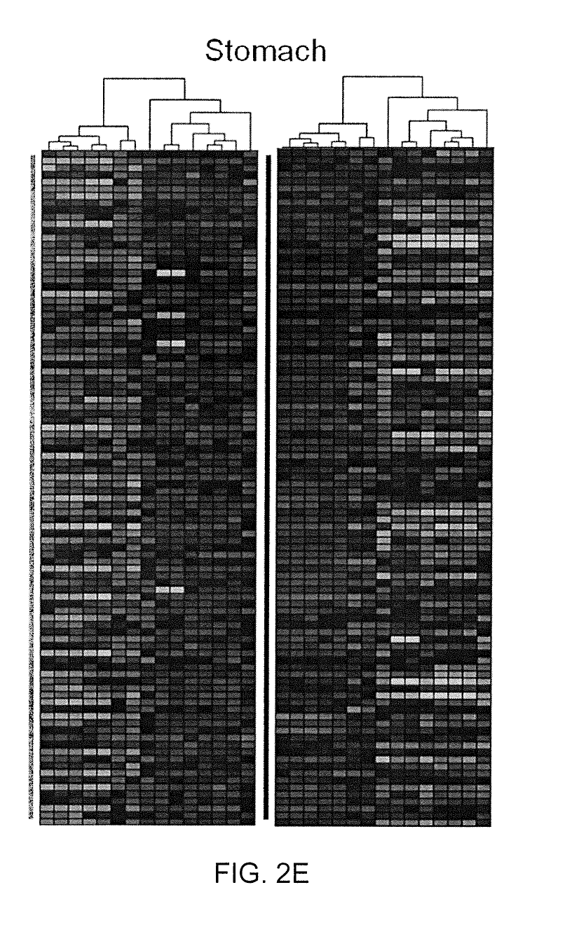

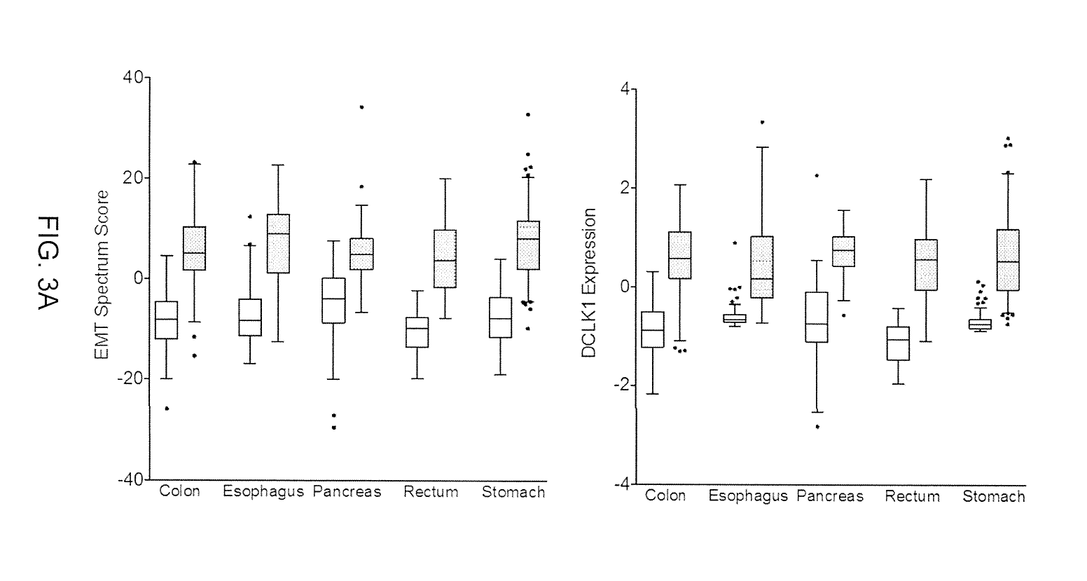

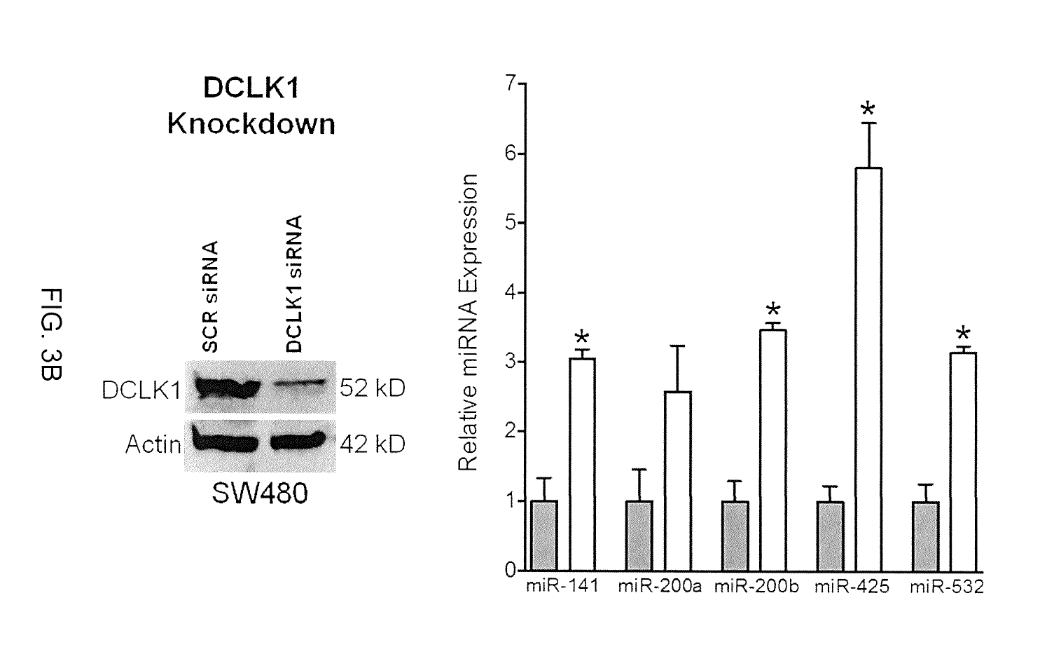

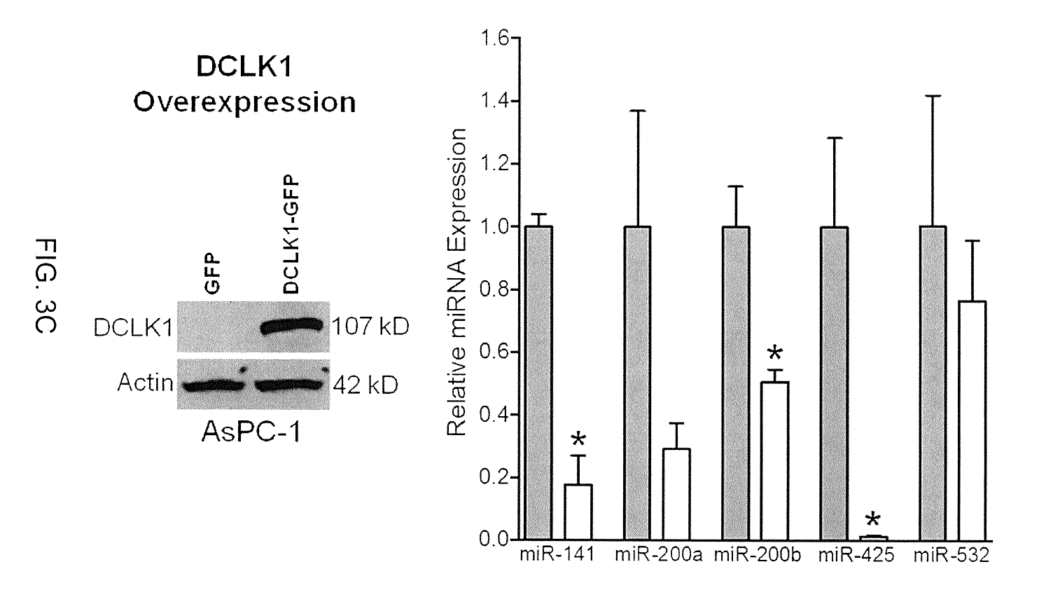

Pearson correlations were performed to determine DCLK1's association with miRNA expression across the 5 tuft-cell containing organ tumors. This analysis revealed a consensus of 15 significantly correlated miRNAs: hsa-miR-99a, hsa-miR-Let7c, hsa-miR-125b-1, hsa-miR-125b-2, hsa-miR-532, hsa-miR-200a, hsa-miR-200b, hsa-miR-429, hsa-miR-425, hsa-miR-218-1, hsa-miR-218-2, hsa-miR-192, hsa-miR-194-2, hsa-miR-100, and hsa-miR-141 (FIG. 1B, Table 1). Comparison of this signature between low DCLK1-expressing (0-25^{th} percentile) and high DCLK1-expressing (75-100^{th} percentile) tumors confirmed the veracity of these findings (FIG. 2A-colon, FIG. 2B-rectum, FIG. 2C-pancreas, FIG. 2D-esophagus, FIG. 2E-stomach). Moreover, high miRNA-signature tumors demonstrated greatly increased levels of epithelial-mesenchymal transition (EMT) as well as DCLK1 expression when compared to low signature tumors (FIG. 3A). In an alternate embodiment the signature can comprise as few as five of the 15 significantly correlated miRNAs, particularly hsa-miR-125b-2, hsa-miR-200a, hsa-miR-125b-1, hsa-miR-99a, and hsa-miR-192.

The derived signature supports previous findings that DCLK1 is both associated with and regulates hsa-miR-200 EMT suppressors [4, 25]. Additionally, we observed changes in expression of 4 key miRNA-clusters including the hsa-miR-99a/125b-2/Let-7c stemness-associated cluster (upregulated); the hsa-miR-200a/200b/429 EMT-suppressor cluster (downregulated); the hsa-miR-192/194-2/200c tumor suppressor and p53-inducer cluster (downregulated); and the hsa-miR-100/125b-2 EMT-inducer cluster (upregulated). Interestingly, the expression of miRNAs that demonstrate shared sequence motifs but distant chromosomal locations were correlated to DCLK1 expression (e.g. hsa-miR-125b-1/2 and hsa-miR-281-1/2) suggesting targeted specificity for DCLK1 or vice-versa. These findings, in consideration of our previously reported studies, suggest that DCLK1 is capable of inducing a stemness and EMT-supporting miRNA signature that may have significant implications in GI tumorigenesis.

To determine whether any of the miRNAs in the signature are directly regulated by DCLK1, we isolated mature miRNAs from SW480 cells, which express high endogenous levels of DCLK1, following transfection with scrambled or DCLK1-targeted siRNA—and from AsPC-1 cells, which express very low levels of DCLK1, stably expressing vector or DCLK1. For both of these sets of cells we isolated proteins to confirm the desired changes in DCLK1 expression. miRNA-specific reverse transcription and real-time PCR revealed that at least 5 of the miRNAs hsa-miR-200a, hsa-miR-200b, hsa-miR-425, and hsa-miR-532 are all upregulated by DCLK1 knockdown (FIG. 3B) and downregulated by DCLK1 overexpression (FIG. 3C) in agreement with their correlation to DCLK1 in the TCGA datasets. These findings indicate that the relationship between the derived miRNA signature and DCLK1 is not merely correlative, but that DCLK1 directly regulates at least one-third of the miRNAs that make up the signature.

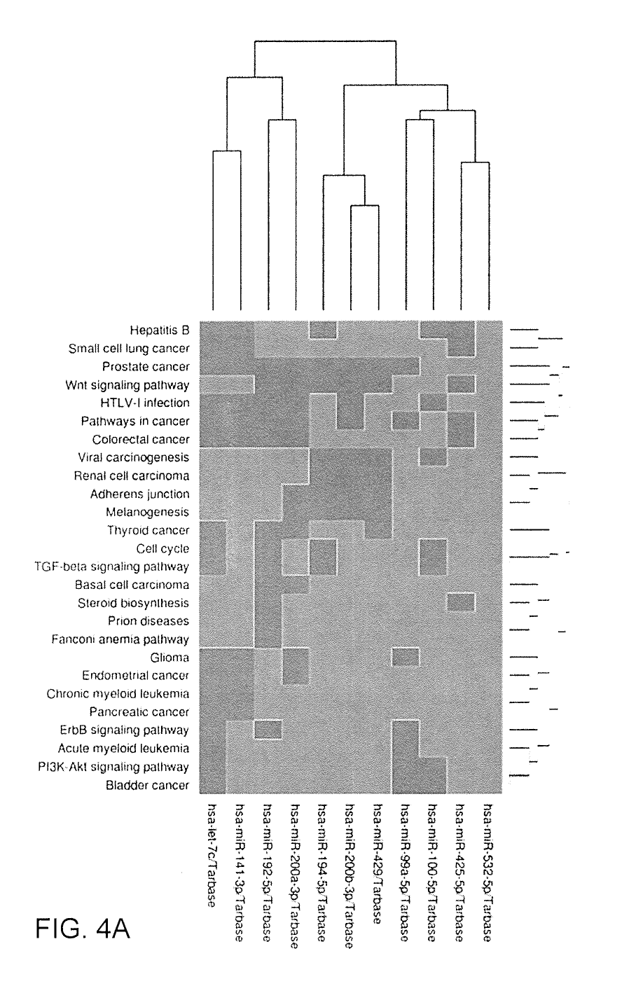

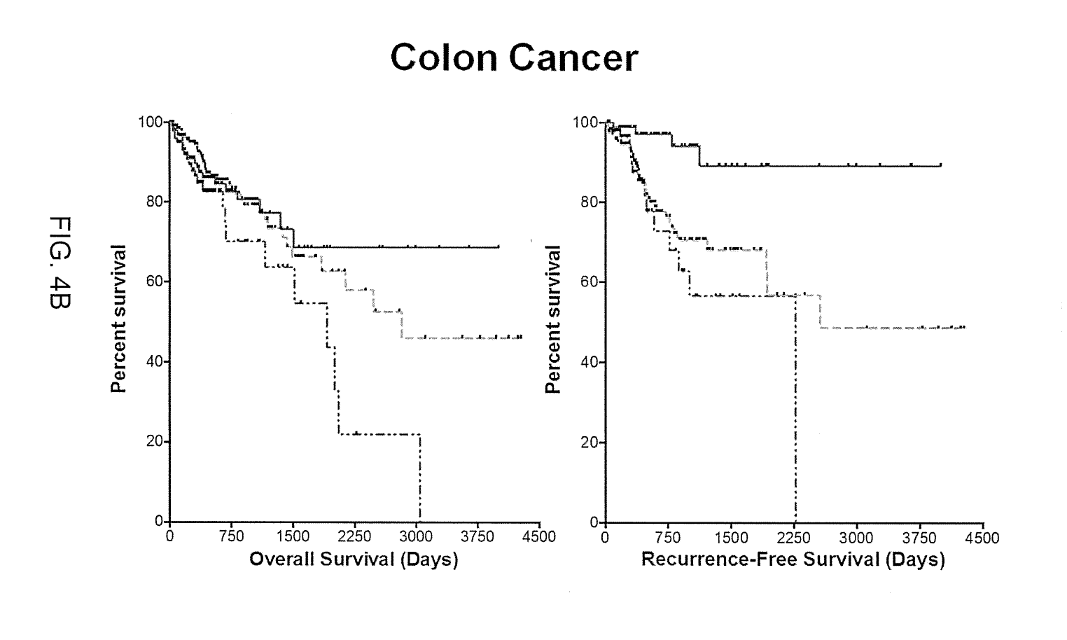

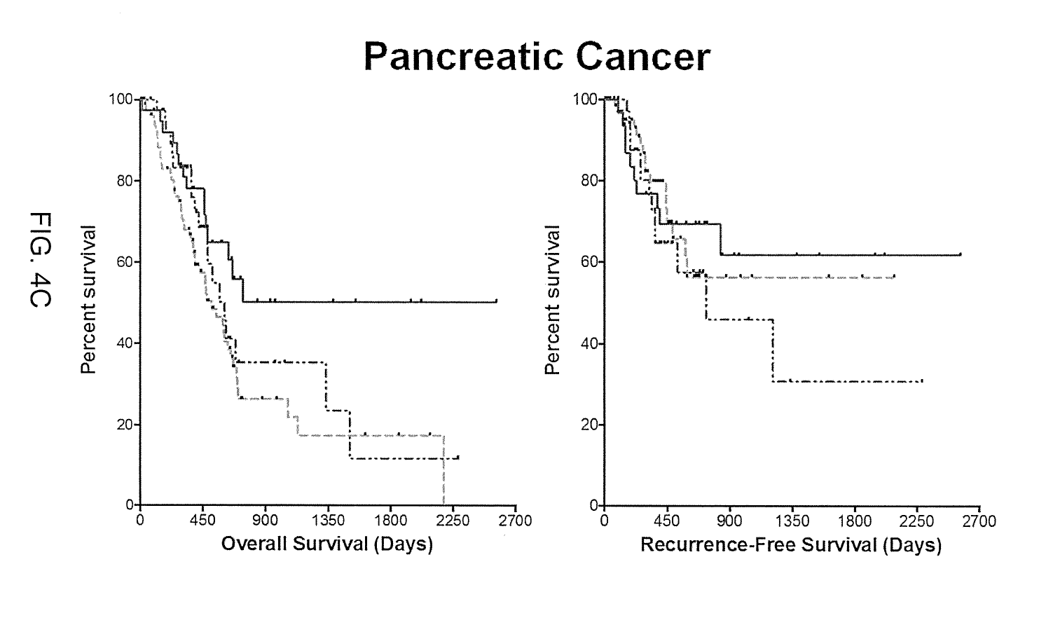

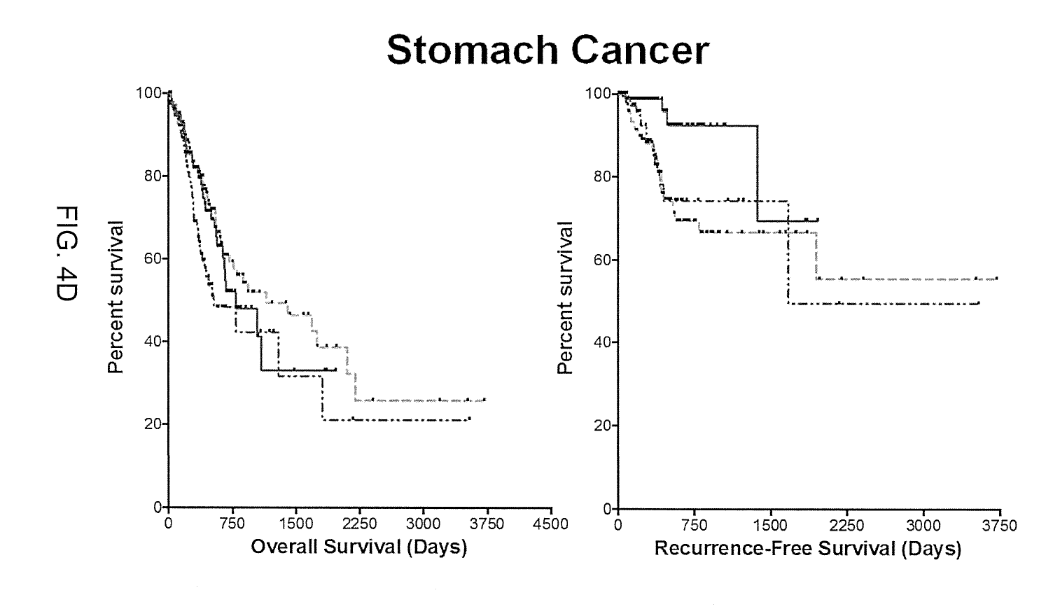

To further assess the potential functional relevance of this DCLK1-specific miRNA signature, we subjected the 15 miRNA signature to KEGG pathway analysis using mirPath (DIANA Tools) with Tarbase as a reference for gene targets. Out of the 15 miRNAs, 11 had gene targets listed in Tarbase. Generation of KEGG pathways based on these targets revealed interesting enrichments for cancer-related pathways in which DCLK1 is known to have functional significance including colorectal, pancreatic, and renal cell cancers. Additionally, important processes that affect tumor initiation and progression such as tight junction-regulating targets and TGF-beta signaling among others were also enriched (FIG. 4A).

A DCLK1-Based 15-miRNA Signature Predicts Survival in Colon and Pancreatic Cancer

Following determination of the miRNA signature and its potential functional significance we sought to determine if the signature could predict survival in any of the 5 studied cancers. An overall signature metric was calculated by summing values for upregulated miRNAs and subtracting values for downregulated miRNAs. Patients were grouped by level of signature expression into low (0-25^{th} percentile), mid (25-75^{th} percentile), and high expression (75-100^{th} percentile). Kaplan-Meier survival analysis demonstrated that the DCLK1-derived miRNA signature could be used to strongly predict both overall and recurrence-free survival in colon cancer. All of the colon cancer patients in the high signature expression group experienced a recurrence of disease by approximately 75 months and no patient survived beyond month 100. In contrast, less than 20% of patients with a low expression signature experienced a recurrence by approximately 150 months and overall survival remained at 70% for this time period (FIG. 4B).

In pancreatic cancer patients, the signature was able to significantly predict overall survival, but not recurrence-free survival. In patients with mid to high level signature expression <15% of patients remained alive after approximately 73 months. However, approximately half of the patients with low signature expression survived to 90 months (FIG. 4C). Although analysis of recurrence-free survival did not reach statistical significance, there was a nearly 25-30% increase of recurrence observed among patients with high signature expression as compared to those with mid and low signature expression (FIG. 4C). The DCLK1-derived miRNA signature was predictive of overall and recurrence-free survival in gastric adenocarcinoma (FIG. 4D).

These data taken together indicate that the 15-miRNA signature presented here as a surrogate for DCLK1 activity in gastrointestinal cancers can serve as a prognostic marker of recurrence, especially gastrointestinal cancers derived from organs with DCLK1-positive tuft cells. In alternate embodiments fewer than 15 miRNAs may be used to calculate the signature, such as 5, 6, 7, 8, 9, 10, 11, 12, 13, or 14 miRNAs from the group of 15 miRNAs described above.

Sub-Group Analysis of the 15-miRNA Survival Signature

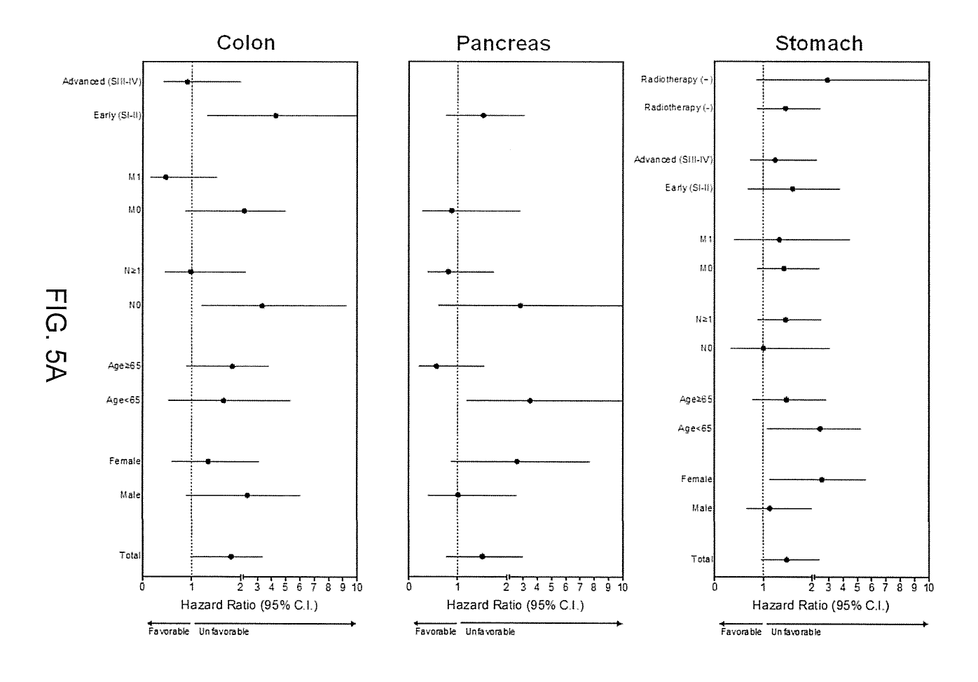

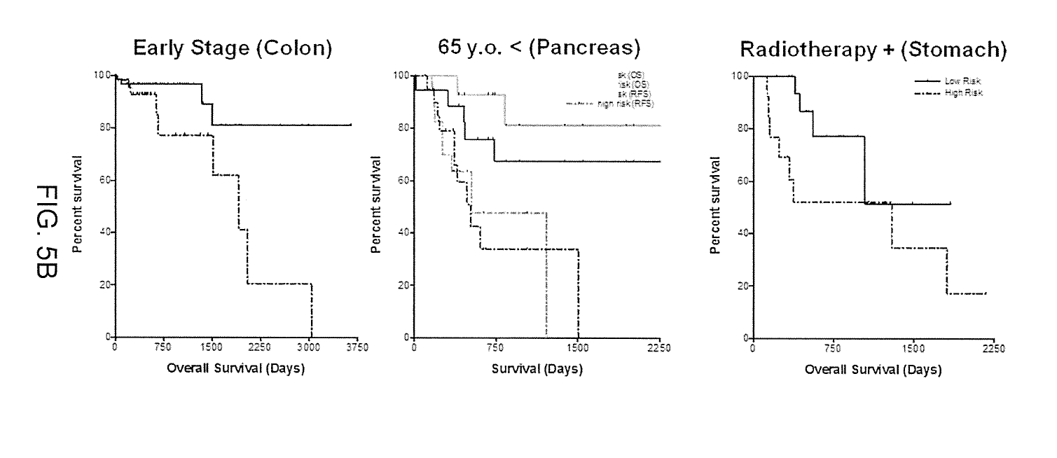

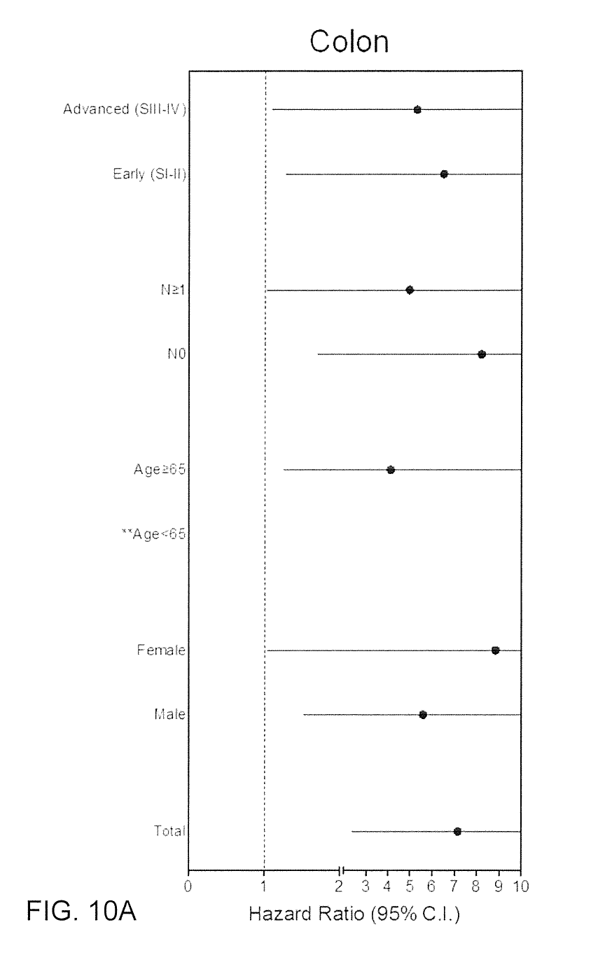

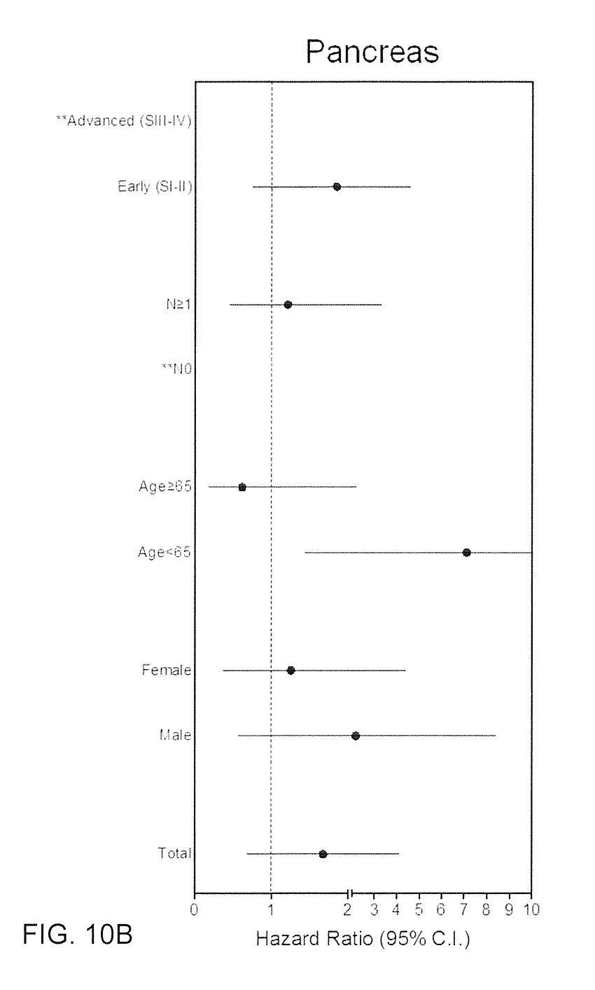

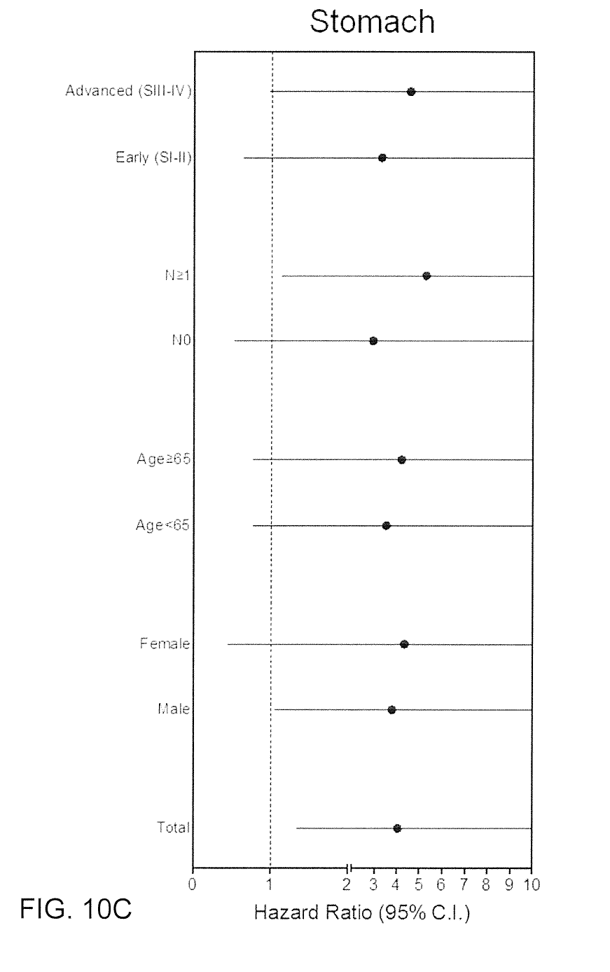

In order to better understand the prognostic significance of the miRNA signature in patients with colon, pancreatic, or gastric cancer, we performed Cox regression analysis on clinical subgroups stratified by low and high-risk miRNA-signature and compared the resulting hazard ratios (FIG. 5A). In colon cancer, early stage patients without signs of nodal or distant metastases who demonstrated high signature expression had a 2-4 fold higher hazard ratio when assessing overall survival. In those with pancreatic cancer, the high-risk signature appeared to be strongly predictive of overall survival in patients under the age of 65, but of limited use in older patients. Finally in gastric cancer patients, high signature expression was consistent for most subgroups assessed, but may be most useful for patients under the age of 65. It also may have some prognostic value for patients undergoing radiation therapy (FIG. 5A), which may be related to DCLK1's tumor stem cell role, as these cells are expected to be resistant to radiation. We confirmed the significance of the miRNA-signature to overall survival in the subgroups described above by Kaplan-Meier analysis (FIG. 5B). Moreover, we performed further subgroup analyses to assess the value of the miRNA-signature in recurrence-free survival and found that the signature was mostly consistent across subgroups and that in pancreatic cancer the signature was again most valuable in patients under the age of 65 (FIG. 10A-C). These data indicate that the miRNA-signature has clinical value in specific subsets of patients.

Observed Survival and Receiver-Operating Characteristics of the 15-miRNA Survival Signature

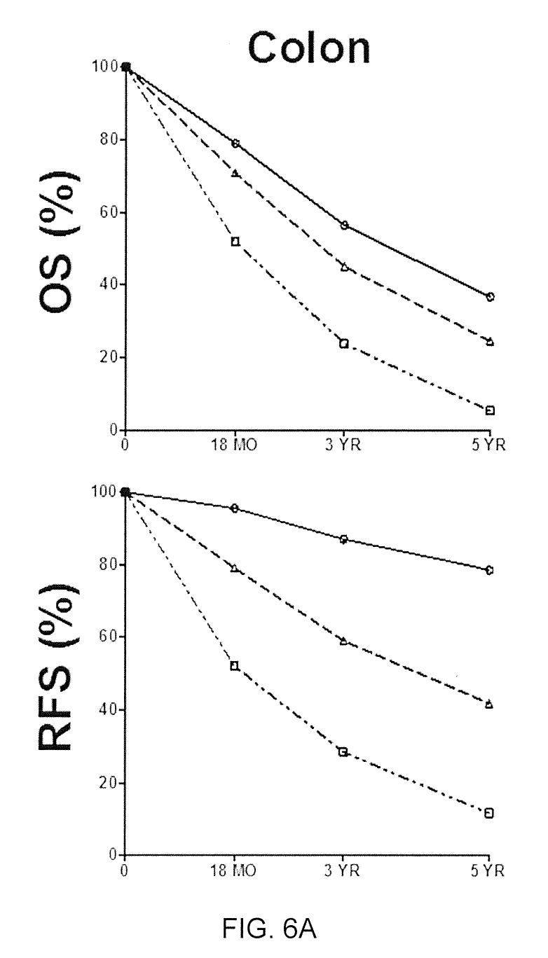

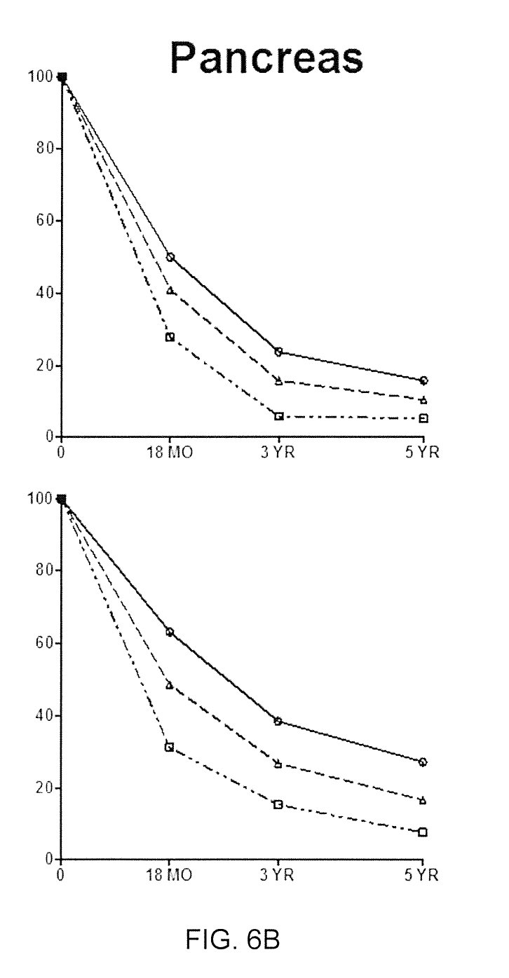

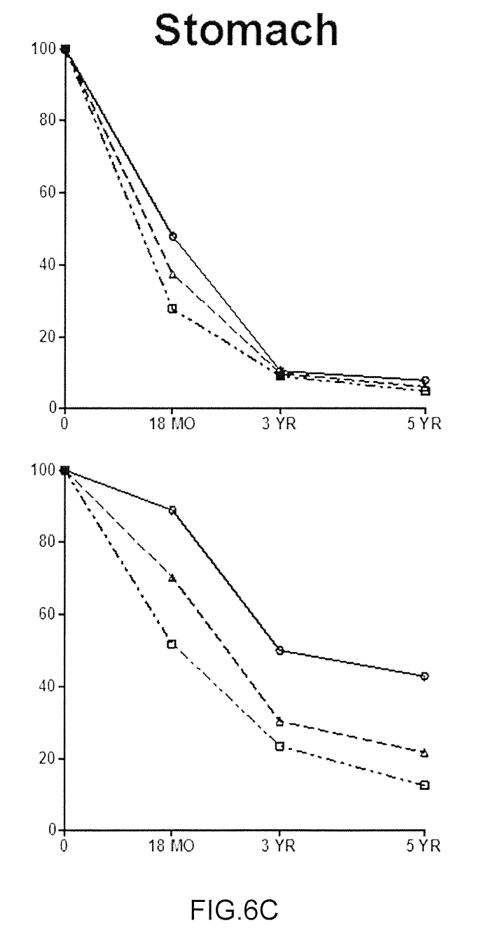

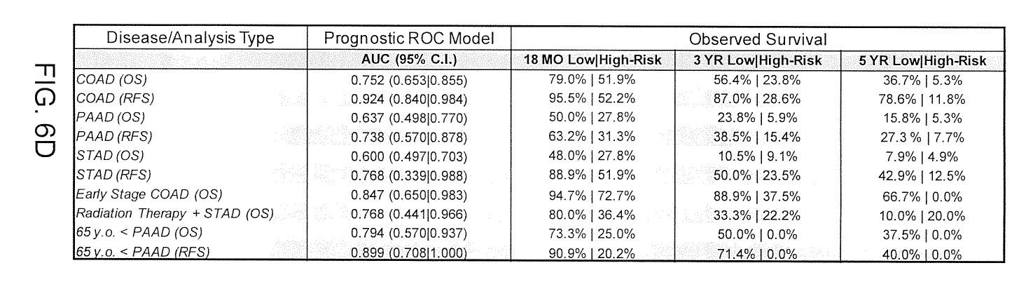

To further assess the signature we divided the patients into groups with definite outcomes at 18 months, 3 years, and 5 years post-diagnosis and determined observed survival percentages. The signature performed well in colon, pancreas, and stomach cancer datasets both in terms of overall and recurrence-free survival (FIG. 6A-C). Patients with the low-risk signature demonstrated better actual survival while patients with the high-risk signature demonstrated poorer actual survival than total. In order to estimate the probable value of the datasets, we utilized the PrognosticROC R package to estimate the probable R.O.C area under the curve (AUC) for the signature (FIG. 6D). Values ranged from approximately 0.65-0.98 in colon cancer, 0.50-0.88 in pancreatic cancer, and 0.34-0.99 in stomach cancer. For the subgroups discussed in FIG. 5, values ranged from approximately 0.44-1. These findings indicate that the miRNA-signature of the present disclosure has significant value as, but is not limited to, a prognostic tool in colon, pancreatic, and stomach cancers and demonstrates that the signature can be used in practice to predict patient risk of death and recurrence.

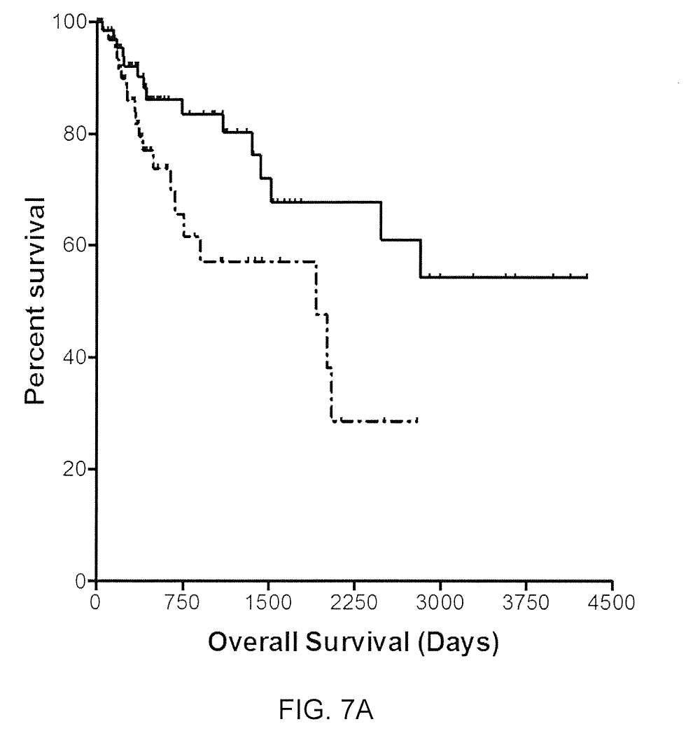

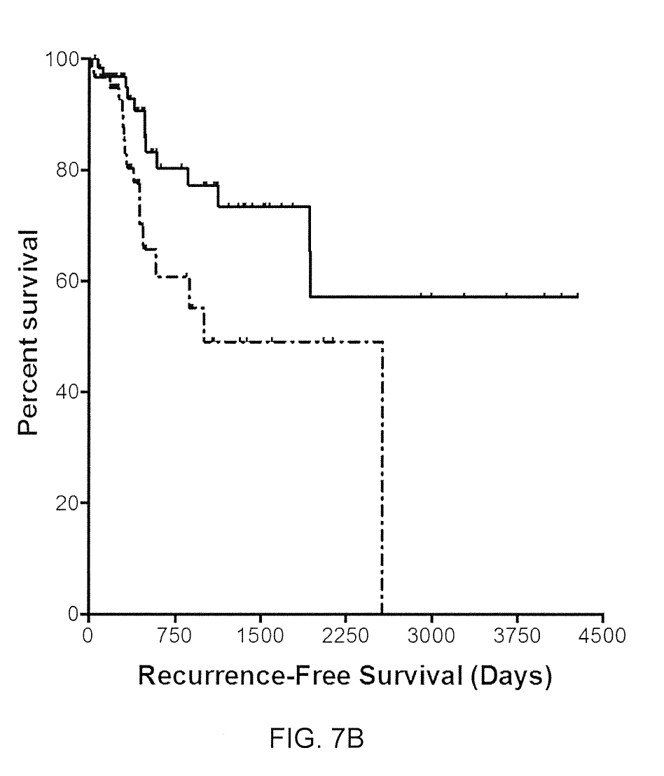

Using Kaplan-Meier and Cox regression analysis, we found that the 15-miRNA signature was able to predict survival in colon, pancreas, and gastric cancers. The signature had the strongest predictive ability in colon cancer where strong data supports a DCLK1+ cell origin for the APC mutant form of this cancer and emerging data suggests a role in the KRAS mutant counterpart [34]. It is notable that the signature was particularly effective at predicting overall survival in patients with early stage (I-II) disease. In fact, early stage patients with high signature expression (HR: 2.751; 95% C.I.: 1.429|11.560) demonstrated survival comparable to patients with advanced stage (III-IV) disease (HR: 2.851; 95% C.I.: 1.879|4.765) as confirmed by Kaplan-Meier analysis (p=0.314). This finding highlights the clinical potential of this miRNA signature, and with further validation its use in identifying high-risk early stage patients that might require more aggressive treatment and follow-up. Additionally, in all stages of disease the high-risk signature predicted a dramatically increased recurrence hazard (>7-fold compared to the low-risk profile). These findings support the role of the DCLK1-based miRNA signature in determining a prognosis for colon cancer patients. We note that a number of groups have demonstrated that DCLK1 expression can predict survival in colon cancer. Although we found similar significant results using DCLK1 gene expression data from the TCGA colon cancer RNA-seq dataset (FIG. 7A-B), the presently disclosed DCLK1-based miRNA signature was able to stratify risk with much greater efficiency.

As in colon cancer, the signature was able to predict overall and recurrence-free survival in gastric cancer. The signature demonstrated better predictive ability in younger (<65 years old) and female patients with a hazard ratio of approximately 3 for these groups. Because gastric cancer is characterized by high associated mortality, predictable biomarkers may greatly improve disease stratification, diagnosis, and treatment protocols. However, attempts to develop biomarkers based on alterations on the gene and protein level have so far failed to produce useful, stable assays for gastric cancer patients. Previous research has shown that female gender and diffuse histopathology are often seen in younger patients with gastric cancer, and that these tumors are molecularly unique and more aggressive than tumors in elderly patients. Screening these patients with the presently disclosed DCLK1-based miRNA signature has the potential to allow clinicians to pursue different treatment strategies in high-risk gastric cancer patients. Another interesting finding was the ability of the signature to predict overall survival in patients receiving radiotherapy. Although the confidence interval for this assessment was wide, this finding may support a stem-like role for DCLK1 in stomach cancer, as cancer stem cells are known to resist radiotherapy.

Finally, despite a relatively small sample size (n=163), the presently disclosed miRNA signature was able to predict overall survival in pancreatic cancer patients. Additionally, there was a trend towards predicting recurrence free survival among the stratified groups, but this did not reach statistical significance, likely due to the small sample size (n=138).

The present findings are novel in that we used the expression of the DCLK1 tumor stem cell marker as a guide to derive a unique, potentially stable miRNA signature that predicts survival in patients with colon, gastric and pancreatic cancer. These results lend support to a potential pan-gastrointestinal role for the DCLK1+ tuft cell and tumor stem cell and the functional significance of DCLK1 in colon, pancreatic, and stomach cancer, as well as other gastrointestinal cancers.

In at least one embodiment, the present disclosure is directed to a primer set (kit) for measuring doublecortin-like kinase 1 (DCLK1) activity in a biological sample, wherein the primer set comprises at least five (i.e., five or more) forward primers, each of which is specific for a different microRNA molecule selected from the group consisting of hsa-miR-99a, hsa-miR-100, hsa-miR-125-B1, hsa-miR-125-B2, hsa-miR-141, hsa-miR-192, hsa-miR-194-2, hsa-miR-200a, hsa-miR-200b, hsa-miR-218-1, hsa-miR-218-2, hsa-miR-425, has-miR-429, and hsa-miR-532, and hsa-miR-let7c (see Table 1). In at least one embodiment, the primer set may comprise forward primers specific for 6, 7, 8, 9, 10, 11, 12, 13, 14, or all 15 of these microRNAs. In at least one embodiment, the different microRNA molecules for which the at least five forward primers are specific include hsa-miR-99a, hsa-miR-125b-1, hsa-miR-125b-2, hsa-miR-192, and hsa-miR-200a. In at least one embodiment of the primer set, at least some of the different microRNA molecules are upregulated and some of the different microRNA molecules are downregulated due to DCLK1 activity. In at least one embodiment, the primer set comprises forward primers specific for microRNAs not listed in Table 1. A typical microRNA exists as a 5′ isoform and as a 3′ isoform. One isoform is usually predominant in a given sample. Each isoform requires a different 5′ or 3′ forward primer for extension. Thus in at least certain non-limiting embodiments, each primer set comprises only one forward primer for each specific target miRNA, e.g., either the 5′ forward primer, or the 3′ forward primer specific for the target miRNA. Examples of such 5′ and 3′ forward primers that can be used in the primer kit and method of use thereof include, but are not limited to, those shown in Tables 2A-B and Tables 3A-B.

TABLE 2A
5′ Forward primers (1-4)

| | | SEQ | | SEQ | | SEQ | | SEQ |
|---|---|---|---|---|---|---|---|---|
| miRNA ID | primer 1 | ID | primer 2 | ID | primer 3 | ID | primer 4 | ID |
| hsa-miR-99a | aacccgtagatccga | 30 | acccgtagatccgat | 31 | cccgtagatccgatc | 32 | ccgtagatccgatct | 33 |
| hsa-miR-100 | aacccgtagatccga | 37 | acccgtagatccgaa | 38 | cccgtagatccgaac | 39 | ccgtagatccgaact | 40 |
| hsa-miR-125b-1 | tccctgagaccctaa | 44 | ccctgagaccctaac | 45 | cctgagaccctaact | 46 | ctgagaccctaactt | 47 |
| hsa-miR-125b-2 | tccctgagaccctaa | 51 | ccctgagaccctaac | 52 | cctgagaccctaact | 53 | ctgagaccctaactt | 54 |
| hsa-miR-141 | catcttccagtacag | 58 | atcttccagtacagt | 59 | tcttccagtacagtg | 60 | cttccagtacagtgt | 61 |
| hsa-miR-192 | ctgacctatgaattg | 65 | tgacctatgaattga | 66 | gacctatgaattgac | 67 | acctatgaattgaca | 68 |
| hsa-miR-194-2 | tgtaacagcaactcc | 72 | gtaacagcaactcca | 73 | taacagcaactccat | 74 | aacagcaactccatg | 75 |
| hsa-miR-200a | catcttaccggacag | 79 | atcttaccggacagt | 80 | tcttaccggacaatg | 81 | cttaccggacagtgc | 82 |
| hsa-miR-200b | catcttactgggcaa | 86 | atcttactgggcagc | 87 | tcttactgggcagca | 88 | cttactgggcagcat | 89 |
| hsa-miR-218-1 | ttgtgcttgatctaa | 93 | tgtacttgatctaac | 94 | gtgcttgatctaacc | 95 | tgcttgatctaacca | 96 |
| hsa-miR-218-2 | ttgtgcttgatctaa | 100 | tgtgcttgatctaac | 101 | gtgcttgatctaacc | 102 | tgcttgatctaacca | 103 |
| hsa-miR-425 | aatgacacgatcact | 107 | atgacacgatcactc | 108 | tgacacgatcactcc | 109 | gacacgatcactccc | 110 |
| hsa-miR-429 | N/A | | N/A | | N/A | | N/A | |
| hsa-miR-532 | catgccttgagtgta | 114 | atgccttaagtgtag | 115 | tgccttgagtgtagg | 116 | gccttgagtgtagga | 117 |
| hsa-miR-Let7c | tgagatagtagattg | 121 | gaggtagtaggttgt | 122 | aggtagtaggttgta | 123 | ggtagtagattgtat | 124 |

TABLE 2B
5′ Forward primers (5-7)

| miRNA ID | primer 5 | SEQ ID | primer 6 | SEQ ID | primer 7 | SEQ ID |
|---|---|---|---|---|---|---|
| hsa-miR-99a | cgtagatccgatctt | 34 | gtagatccaatcttg | 35 | tagatccgatcttgt | 36 |
| hsa-miR-100 | cgtagatccgaactt | 41 | gtagatccgaacttg | 42 | tagatccgaacttgt | 43 |
| hsa-miR-125b-1 | tgagaccctaacttg | 48 | gagaccctaacttgt | 49 | agaccctaacttgtg | 50 |
| hsa-miR-125b-2 | tgagaccctaacttg | 55 | gagaccctaacttgt | 56 | agaccctaacttgtg | 57 |
| hsa-miR-141 | ttccagtacagtgtt | 62 | tccagtacagtgttg | 63 | ccagtacagtattgg | 64 |
| hsa-miR-192 | cctatgaattgacag | 69 | ctatgaattgacagc | 70 | tatgaattgacagcc | 71 |
| hsa-miR-194-2 | acagcaactccatgt | 76 | cagcaactccatgtg | 77 | agcaactccatgtgg | 78 |
| hsa-miR-200a | ttaccggacagtgct | 83 | taccggacagtgctg | 84 | accggacagtgctg | 85 |
| hsa-miR-200b | ttactgggcagcatt | 90 | tactgggcagcattg | 91 | actgggcagcattgg | 92 |
| hsa-miR-218-1 | gcttgatctaaccat | 97 | cttgatctaaccatg | 98 | ttgatctaaccatgt | 99 |
| hsa-miR-218-2 | gcttgatctaaccat | 104 | cttgatctaaccatg | 105 | ttgatctaaccatgt | 106 |
| hsa-miR-425 | acacgatcactcccg | 111 | cacgatcactcccgt | 112 | acgatcactcccgtt | 113 |
| hsa-miR-429 | N/A | | N/A | | N/A | |
| hsa-miR-532 | ccttgagtgtaggac | 118 | cttgagtgtaggacc | 119 | ttgagtgtaggaccg | 120 |
| hsa-miR-Let7c | gtagtaggttgtatg | 125 | tagtaggttgtatgg | 126 | agtaggttgtatgat | 127 |

TABLE 3A
3′ Forward primers (1-4)

| | | SEQ | | SEQ | | SEQ | | SEQ |
|---|---|---|---|---|---|---|---|---|
| miRNA ID | primer 1 | ID | primer 2 | ID | primer 3 | ID | primer 4 | ID |
| hsa-miR-99a | caagctcgcttctat | 128 | aagctcgcttctatg | 129 | agctcgcttctatgg | 130 | gctcgcttctatggg | 131 |
| hsa-miR-100 | caagcttgtatctat | 135 | aagcttgtatctata | 136 | agcttgtatctatag | 137 | gcttgtatctatagg | 138 |
| hsa-miR-125b-1 | acgggttagactctt | 142 | cgggttaggctcttg | 143 | gggttaggctcttgg | 144 | gattaggctcttggg | 145 |
| hsa-miR-125b-2 | tcacaagtcaggctc | 149 | cacaagtcaggctct | 150 | acaagtcaggctctt | 151 | caagtcaaactcttg | 152 |
| hsa-miR-141 | taacactgtctggta | 156 | aacactgtctggtaa | 157 | acactgtctggtaaa | 158 | cactgtctagtaaag | 159 |
| hsa-miR-192 | ctgccaattccatag | 163 | tgccaattccatagg | 164 | gccaattccataggt | 165 | ccaattccataggtc | 166 |
| hsa-miR-194-2 | ccagtggggctgctg | 170 | cagtgaagctgctat | 171 | agtggggctgctgtt | 172 | gtggggctgctgtta | 173 |
| hsa-miR-200a | taacactgtctaata | 177 | aacactgtctggtaa | 178 | acactgtctggtaac | 179 | cactgtctggtaacg | 180 |
| hsa-miR-200b | taatactgcctagta | 184 | aatactgcctggtaa | 185 | atactgcctggtaat | 186 | tactgcctggtaata | 187 |
| hsa-miR-218-1 | atggttccgtcaagc | 191 | tgattccgtcaagca | 192 | ggttccgtcaagcac | 193 | gttccatcaagcacc | 194 |
| hsa-miR-218-2 | catggttctgtcaag | 198 | atggttctgtcaagc | 199 | tggttctgtcaaaca | 200 | agttctgtcaaacac | 201 |
| hsa-miR-425 | atcggaaatatcgtg | 205 | tcgggaatgtcgtgt | 206 | cgggaatgtcgtgtc | 207 | gggaatgtcgtgtcc | 208 |
| hsa-miR-429 | taatactatctggta | 212 | aatactgtctggtaa | 213 | atactgtctggtaaa | 214 | tactatctggtaaaa | 215 |
| hsa-miR-532 | cctcccacacccaag | 219 | ctcccacacccaagg | 220 | tcccacacccaaggc | 221 | cccacacccaaggt | 222 |
| hsa-miR-Let7c | ctgtacaaccttcta | 226 | tgtacaaccttctag | 227 | gtacaaccttctagc | 228 | tacaaccttctagct | 229 |

TABLE 3B
5′ Forward primers (5-7)

| miRNA ID | primer 5 | SEQ ID | primer 6 | SEQ ID | primer 7 | SEQ ID |
|---|---|---|---|---|---|---|
| hsa-miR-99a | ctcgcttctatgggt | 132 | tcgcttctatgggtc | 133 | cgcttctatgggtct | 134 |
| hsa-miR-100 | cttgtatctataggt | 139 | ttgtatctataggta | 140 | tgtatctataggtat | 141 |
| hsa-miR-125b-1 | gttaggctcttggga | 146 | ttaggctcttgggag | 147 | taggctcttgggagc | 148 |
| hsa-miR-125b-2 | aagtcaggctcttgg | 153 | agtcaggctcttggg | 154 | gtcaggctcttggga | 155 |
| hsa-miR-141 | actgtctggtaaaga | 160 | ctgtctggtaaagat | 161 | tgtctggtaaagatg | 162 |
| hsa-miR-192 | caattccataggtca | 167 | aattccataggtcac | 168 | attccataggtcaca | 169 |
| hsa-miR-194-2 | tggggctgctgttat | 174 | ggggctgctgttatc | 175 | aggctgctgttatct | 176 |
| hsa-miR-200a | actgtctggtaacga | 181 | ctgtctggtaacgat | 182 | tgtctggtaacgatg | 183 |
| hsa-miR-200b | actgcctggtaatga | 188 | ctacctggtaatgat | 189 | tgcctggtaatgatg | 190 |
| hsa-miR-218-1 | ttccgtcaagcacca | 195 | tccgtcaagcaccat | 196 | ccgtcaagcaccatg | 197 |
| hsa-miR-218-2 | gttctgtcaagcacc | 202 | ttctgtcaagcaccg | 203 | tctgtcaagcaccgc | 204 |
| hsa-miR-425 | ggaatgtcgtgtcca | 209 | gaatgtcgtgtccgc | 210 | aatgtcgtgtccgcc | 211 |
| hsa-miR-429 | actgtctggtaaaac | 216 | ctgtctggtaaaacc | 217 | tgtctggtaaaaccg | 218 |
| hsa-miR-532 | ccacacccaaggctt | 223 | cacacccaaggcttg | 224 | acacccaaggcttgc | 225 |
| hsa-miR-Let7c | acaaccttctagctt | 230 | caaccttctagcttt | 231 | aaccttctagctttc | 232 |

In at least one other embodiment, the present disclosure is directed to a cDNA or RNA hybridization array for measuring doublecortin-like kinase 1 (DCLK1) activity in a biological sample, wherein the array comprises at least five (i.e., five or more) probes, each of which is specific for a different microRNA molecule selected from the group consisting of hsa-miR-99a, hsa-miR-100, hsa-miR-125-B1, hsa-miR-125-B2, hsa-miR-141, hsa-miR-192, hsa-miR-194-2, hsa-miR-200a, hsa-miR-200b, hsa-miR-218-1, hsa-miR-218-2, hsa-miR-425, has-miR-429, and hsa-miR-532, and hsa-miR-let7c (see Table 1). In at least one embodiment, the array may comprise probes specific for 6, 7, 8, 9, 10, 11, 12, 13, 14, or all 15 of these microRNAs. Non-limiting examples of such probes include the DNA sequences shown in Tables 2A, 2B, 3A and 3B. In at least one embodiment, the different microRNA molecules for which the at least five probes are specific include hsa-miR-99a, hsa-miR-125b-1, hsa-miR-125b-2, hsa-miR-192, and hsa-miR-200a. In at least one embodiment of the array, at least some of the different microRNA molecules are upregulated and some of the different microRNA molecules are downregulated due to DCLK1 activity. In at least one embodiment, the array may comprise probes specific for microRNAs not listed in Table 1.

In at least one other embodiment of a hybridization array, the present disclosure is directed to an miRNA hybridization array for measuring doublecortin-like kinase 1 (DCLK1) activity in a biological sample, wherein the array comprises at least five (i.e., five or more) probes, each of which is specific for a different microRNA molecule selected from the group consisting of hsa-miR-99a, hsa-miR-100, hsa-miR-125-B1, hsa-miR-125-B2, hsa-miR-141, hsa-miR-192, hsa-miR-194-2, hsa-miR-200a, hsa-miR-200b, hsa-miR-218-1, hsa-miR-218-2, hsa-miR-425, has-miR-429, and hsa-miR-532, and hsa-miR-let7c (see Table 1). In at least one embodiment, the array may comprise probes specific for 6, 7, 8, 9, 10, 11, 12, 13, 14, or all 15 of these microRNAs of Table 1. In at least one embodiment, the different microRNA molecules for which the at least five probes are specific include hsa-miR-99a, hsa-miR-125b-1, hsa-miR-125b-2, hsa-miR-192, and hsa-miR-200a. In at least one embodiment of the array, at least some of the different microRNA molecules are upregulated and some of the different microRNA molecules are downregulated due to DCLK1 activity. The probes may be antisense versions of the miRNAs in Table 1. In at least one embodiment, the array may comprise probes specific for miRNAs not listed in Table 1.

In at least one embodiment, the present disclosure is directed to a method of measuring doublecortin-like kinase 1 (DCLK1) activity in a biological sample, comprising, performing a real-time reverse-transcriptase polymerase chain reaction (RT-PCR) on the biological sample using forward primers specific for at least five different microRNA molecules selected from the group consisting of hsa-miR-99a, hsa-miR-100, hsa-miR-125-B1, hsa-miR-125-B2, hsa-miR-141, hsa-miR-192, hsa-miR-194-2, hsa-miR-200a, hsa-miR-200b, hsa-miR-218-1, hsa-miR-218-2, hsa-miR-425, has-miR-429, hsa-miR-532, and hsa-miR-let7c, thereby generating complementary DNA (cDNA) molecules for each of the at least five different microRNA molecules; and detecting the cDNA molecules to determine an expression profile of the at least five different microRNA molecules as a measure of DCLK1 activity in the biological sample. In the method, the different microRNA molecules for which the at least five forward primers are specific may include hsa-miR-99a, hsa-miR-125b-1, hsa-miR-125b-2, hsa-miR-192, and hsa-miR-200a. In the method at least some of the different microRNA molecules may be upregulated and some of the different microRNA molecules are downregulated due to DCLK1 activity. In the method, the expression profile may be used to determine a risk for recurrence of or survival from a gastrointestinal cancer in a subject from whom the biological sample was obtained. The gastrointestinal cancer in the subject may be at least one of colon, pancreas, stomach, uterine, ovarian. bladder, lung, rectal, and esophageal cancer. The method may use Equation I to determine a score based on the expression profile obtained from quantification of the cDNA molecules.

### EXAMPLES

Certain novel embodiments of the present disclosure, having now been generally described, will be more readily understood by reference to the following examples, which are included merely for purposes of illustration of certain aspects and embodiments of the present disclosure, and are not intended to be limiting. The following examples are to be construed, as noted above, only as illustrative, and not as limiting of the present disclosure in any way whatsoever. Those skilled in the art will promptly recognize appropriate variations from the various compositions, structures, components, procedures and methods.

### Example 1

In one embodiment, one or more biological samples are collected from a patient (subject). Mature miRNAs are isolated from patient samples via standard techniques such as trizol precipitation or with the use of a kit for this purpose (e.g., Qiagen miRNeasy isolation kit). The concentrations of the total isolated RNAs are quantified via standard techniques (e.g., nanodrop, OD260/280 on a spectrophotometer, or other techniques). The isolated RNAs are then diluted to equal concentrations and subjected to polyadenylation followed by reverse transcription. An example of the conditions for this process in a thermocycler may include polyadenylation at 37° C. for 30 minutes followed by reverse transcription at 70° C. for 60 min followed by denaturation of the unreacted products at 95° C. for 5 minutes. The reverse transcribed cDNA product is then subjected to real-time PCR using a universal primer for the added poly-A region and a specific primer (5′ forward primer and/or 3′ forward primer) for the mature miRNA. An example of the conditions for this process in a thermocycler may include initial denaturation at 95° C. for 3 min, followed by 40 cycles of 15 seconds denaturation at 95° C., 20 seconds of annealing at 60° C., and 15 seconds of extension at 72° C. Additionally the final products may be quantified for quality by melt curve analysis and for expression by standard ethidium bromide gel imaging or similar techniques. Moreover, the principles described here for detection of the miRNAs may be applicable, for example, to a 96 well plate precoated with reagents necessary to perform all of these steps at once, or to perform the real-time PCR portion of the procedure. For example, for real-time PCR a 96 well plate may be precoated with Taq polymerase, dNTPs, PCR buffer salts, universal reverse miRNA primer, and specific miRNA primer. Diluted miRNA-derived cDNAs may be added directly to this plate to perform real-time PCR. Additionally, this plate may contain spiked standards or standards derived from archived specimens that allow the assessment of relative miRNA expression level. Moreover, it will contain housekeeping gene specific primers that allow normalization of the resulting Ct values via a method like the delta-delta Ct method. For example, primers against U6 may be used for this purpose.

### Example 2

Instead of the primer and PCR quantification method of the miRNAs of Table 1 as described above, alternate embodiments of the sets, kits, arrays, and methods of the present disclosure can utilize probe arrays, such as a cDNA hybridization array or an miRNA hybridization array, for quantification of miRNAs.

A cDNA hybridization array can utilize probes comprising the same nucleic acid sequences as the forward primers utilized above in the PCR method (e.g., see Tables 2A-3B), or any oligonucleotide sequences which hybridize with high specificity to the microRNAs of the present disclosure to form probe-miRNA complexes. Each probe generally also comprises one or more fluorescent, luminescent or chemiluminescent label or reporter group linked thereto for quantification of the probe-miRNA complex. Examples of such labels are described elsewhere herein, and still other such labels will readily come to mind of a person having ordinary skill in the art. An miRNA hybdridization array can utilize probe(s) comprising the antisense complementary sequence of an untranscribed miRNA sequence and may comprise a molecule such as streptavidin. In the method, miRNA molecules isolated from a subject sample are covalently liked to a fluorescent, luminescent or chemiluminescent label or reporter group, e.g., a fluorophore. A substrate (e.g., an array platform) comprising adhered probe molecules specific for one or more particular miRNAs is provided. The miRNA molecules with the linked fluorophore are applied to the probe-bearing array platform which is processed to measure the presence and quantity of particular miRNA molecules that are present in the sample. The probe molecules may be DNA primer molecules having specificity for the miRNA sequences, or antisense RNA sequences with complementarity to all or portions of the miRNA sequences and which hybridize thereto.

For example, in the case of a cDNA hybridization array, for the hsa-miR-99a 5′ isoform sequence AACCCGUAGAUCCGAUCUUGUG (SEQ ID NO:1), the probe oligonucleotide sequence in a cDNA hybridization array can be AACCCGTAGATCCGA (SEQ ID NO:30) which is the same as the forward primer in the PCR method described above. This sequence binds the reverse complement product, TTGGGCATCTAGGCA (SEQ ID NO:233), which is the product of the PCR process. Any of the other primer sequences could be utilized accordingly.

Alternatively, in the case of an miRNA hybridization array, for the hsa-miR-99a 5′ isoform sequence AACCCGUAGAUCCGAUCUUGUG (SEQ ID NO:1), the probe sequence could be CACAAGAUCGGAUCUA (SEQ ID NO:234), which is the antisense (reverse) complement of a portion of the miRNA sequence. As noted above, the RNA oligonucleotide sequence which functions as an miRNA probe could be any sequence which hybridizes with high specificity to a particular miRNA. The present disclosure therefore explicitly incorporates by reference all RNA antisense complementary sequences of each entire miRNA sequence in Table 1, i.e., of SEQ ID NOS: 1-29, and of DNA sequences 30-232 in Tables 2A, 2B, 3A, and 3C. The present disclosure further explicitly incorporates by reference all RNA antisense complementary sequences of each subportion of miRNA sequences SEQ ID NOS: 1-29 which comprise at least 12, 13, 14, 15, 16, 17, 18, 19, 20, or 21 contiguous nucleotides thereof. For example CACAAGAUCGGAUCUA (SEQ ID NO:234) is an antisense RNA sequence of a 16-mer subportion of AACCCGUAGAUCCGAUCUUGUG (SEQ ID NO:1).

### Example 3

A hypothetical example of the calculation of a risk score using the presently disclosed miRNA signature is below.

A biopsy from a Stage I colon cancer patient is obtained from the clinic. The biopsy is lysed and the small RNAs are isolated using the Qiagen miRNeasy kit. The amounts of RNAs present are quantified in a quartz microcuvette using a spectrophotometer (OD260/280). Equal amounts of total small RNAs are added to a PCR tube containing 1 microliter of polyadenylase, 1 microliter of reverse-transcriptase, 1 microliter of random hexamers, and 1 microliter of dNTPs and the mixture is dilute with molecular grade water to a final volume of 20 microliters. The reaction mixtures are then placed in a thermocycler with program settings for 37° C. for 30 minutes (polyadylation), 70° C. for 60 minutes (reverse-transcription), 95° C. for 5 minutes (denaturation of unreacted products), and an indefinite hold at 4° C. to protect the cDNA product from degradation. Following this process the 1 microliter of the cDNA product is added to 15 wells of a 96 well plate where each well contains lyophilized universal reverse primer (for the Poly-A region), specific primer for 1 of the 15 miRNAs in the signature, PCR buffer salts, dNTP salts, Taq polymerase, and SYBR green detection reagent. Additional wells contain the same components but with housekeeping gene specific primers (e.g. U6). Additionally, pre-determined standards either spike-in or from archived specimens are added to separate sets of wells with the same components. Following addition of the cDNAs the total volume of each well is brought up to 20 microliters and the plates is sealed with photo-transparent film and placed in a real-time PCR thermocycler. The plate is cycled using the following procedure: 95° C. initial denaturation for 3 minutes, followed by 40 cycles of 15 seconds denaturation at 95° C., 20 seconds of annealing at 60° C., and 15 seconds of extension at 72° C. The resulting Ct threshold values are calculated for the analyte sample from the Stage I colon cancer patient tumor and from the pre-determined standards for each of the 15 microRNAs normalized to the U6 housekeeping gene to obtain fold change via the delta-delta Ct method. The resulting values are then input into Equation I:

$$\frac{\begin{matrix}\sum Fold change in Upregulated miRNAs - \\ \sum Fold change in Downregulated miRNAs\end{matrix}}{total number of types of miRNAs in signature}$$

where:

Upregulated hsa-miRNAs=hsa-miR-100, hsa-miR-125-B1, hsa-miR-125-B2, hsa-miR-let7c, hsa-miR-99a, hsa-miR-218-1, and hsa-miR-218-2, and Downregulated hsa-miRNAs=hsa-miR-141, hsa-miR-192, hsa-miR-194-2, hsa-miR-200a, hsa-miR-200b, hsa-miR-425, has-miR-429, and hsa-miR-532.

The units are relative units which are “fold change” determined by normalizing from the housekeeping gene. However, with other techniques such as hybridization it is possible to calculate the signature from raw numbers. As an example for the real time PCR scenario described herein hypothetical results include:

### Fold Change Relative to Standards

Upregulated miRNAs:
miR-100: 2.1; miR-125-B1: 3.2; miR-125-B2: 0.3; miR-let7c: −1.2; miR-99a: 4.3; miR-218-1: 7.2; miR-218-2: 1.7.
Downregulated miRNAs:
miR-141: −1.3; miR-192: 0.4; miR-194-2: −4.2; miR-200a: 0.1; miR-200b: 0.0; miR-425: −3.1; miR-429: 1.2; miR-532: −6.3.
Eqn. I is used to calculate a score:

(Sum[Upregulated]−Sum[Downregulated])/total number of types of miRNAs

[2.1+3.2+0.3+(−1.2)+4.3+7.2+1.7]−[−1.3+0.4+(−4.2)+0.1+0.0+(−3.1)+1.2+(−6.3)]=[17.6]−[−13.2]=30.8

30.8/n genes=30.8/15=2.05333=Risk score

A positive score would indicate increased risk of recurrence and death. A score of 0 would indicate no change in risk from the average patient. A negative score would indicate a decreased risk compared to the average patient. All of the scores would be in reference to established thresholds from analytical standards—either artificially introduced synthetic miRNAs (i.e. spike-in) or from archived patient biopsies.

It will be understood from the foregoing description that various modifications and changes may be made in the various embodiments of the present disclosure without departing from their true spirit. The description provided herein is intended for purposes of illustration only and is not intended to be construed in a limiting sense, except where specifically indicated. Thus, while the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulation of the various components and compositions described herein, the methods described herein or in the steps or the sequence of steps of the methods described herein without departing from the spirit and scope of the present disclosure. All patents, published patent applications, and non-patent publications referenced in any portion of this application are herein expressly incorporated by reference in their entirety to the same extent as if each individual patent or publication was specifically and individually indicated to be incorporated by reference.

1. A primer set for measuring doublecortin-like kinase 1 (DCLK1) activity in a biological sample, comprising:
at least five forward primers, each forward primer specific for a different microRNA molecule, the different microRNA molecules selected from the group consisting of hsa-miR-99a, hsa-miR-100, hsa-miR-125-B1, hsa-miR-125-B2, hsa-miR-141, hsa-miR-192, hsa-miR-194-2, hsa-miR-200a, hsa-miR-200b, hsa-miR-218-1, hsa-miR-218-2, hsa-miR-425, has-miR-429, and hsa-miR-532, and hsa-miR-let7c.

2. The primer set of claim 1, wherein the different microRNA molecules for which the at least five forward primers are specific include hsa-miR-99a, hsa-miR-125b-1, hsa-miR-125b-2, hsa-miR-192, and hsa-miR-200a.

3. The primer set of claim 1, wherein each forward primer is specific for a 5′ isoform sequence of one of said microRNA molecules or for a 3′ isoform sequence of one of said microRNA molecules.

4. The primer set of claim 1, wherein at least some of the different microRNA molecules are upregulated and some of the different microRNA molecules are downregulated due to DCLK1 activity.

5. The primer set of claim 1, wherein each of the at least five forward primers is disposed in a reaction mixture.

6. A hybridization array for measuring doublecortin-like kinase 1 (DCLK1) activity in a biological sample, comprising:
at least five oligonucleotide probes, each probe specific for a different microRNA molecule, the different microRNA molecules selected from the group consisting of hsa-miR-99a, hsa-miR-100, hsa-miR-125-B1, hsa-miR-125-B2, hsa-miR-141, hsa-miR-192, hsa-miR-194-2, hsa-miR-200a, hsa-miR-200b, hsa-miR-218-1, hsa-miR-218-2, hsa-miR-425, has-miR-429, and hsa-miR-532, and hsa-miR-let7c.

7. The hybridization array of claim 6, wherein the different microRNA molecules for which the at least five probes are specific include hsa-miR-99a, hsa-miR-125b-1, hsa-miR-125b-2, hsa-miR-192, and hsa-miR-200a.

8. The hybridization array of claim 6, wherein each probe is specific for a 5′ isoform sequence of one of said microRNA molecules or for a 3′ isoform sequence of one of said microRNA molecules.

9. The hybridization array of claim 6, wherein at least some of the different microRNA molecules are upregulated and some of the different microRNA molecules are downregulated due to DCLK1 activity.

10. The hybridization array of claim 6, wherein the at least five probes are immobilized on a surface.

11. The hybridization array of claim 10, wherein the surface comprises one or more microarray plates.

12. The hybridization array of claim 10, wherein the surface comprises a plurality of microbeads.

13. The hybridization array of claim 6, comprising a cDNA hybridization array wherein the probes comprise DNA.

14. The hybridization array of claim 6, comprising an RNA hybridization array wherein the probes comprise RNA.

15. A method of measuring doublecortin-like kinase 1 (DCLK1) activity in a biological sample, comprising:
performing a real-time reverse-transcriptase polymerase chain reaction (RT-PCR) on the biological sample using forward primers specific for at least five different microRNA molecules selected from the group consisting of hsa-miR-99a, hsa-miR-100, hsa-miR-125-B1, hsa-miR-125-B2, hsa-miR-141, hsa-miR-192, hsa-miR-194-2, hsa-miR-200a, hsa-miR-200b, hsa-miR-218-1, hsa-miR-218-2, hsa-miR-425, has-miR-429, hsa-miR-532, and hsa-miR-let7c, thereby generating complementary DNA (cDNA) molecules for each of the at least five different microRNA molecules; and
detecting the cDNA molecules to determine an expression profile of the at least five different microRNA molecules as a measure of DCLK1 activity in the biological sample.

16. The method of claim 15, wherein the different microRNA molecules for which the at least five forward primers are specific include hsa-miR-99a, hsa-miR-125b-1, hsa-miR-125b-2, hsa-miR-192, and hsa-miR-200a.

17. The method of claim 15, wherein each of the at least five forward primers is specific for a 5′ isoform sequence of one of said microRNA molecules or for a 3′ isoform sequence of one of said microRNA molecules.

18. The method of claim 15, wherein at least some of the different microRNA molecules are upregulated and some of the different microRNA molecules are downregulated due to DCLK1 activity.

19. The method of claim 15, wherein the expression profile is used to determine a risk for recurrence of or survival from a cancer in a subject from whom the biological sample was obtained.

20. The method of claim 19, wherein the cancer in the subject is at least one of colon, pancreas, stomach, uterine, ovarian, bladder, lung, rectal, and esophageal cancer.

21-34. (canceled)


SEQUENCE LISTING (234 sequences)

SEQ ID NO: 1 - RNA, 22 nt, Homo sapiens
aacccguagauccgaucuugug

SEQ ID NO: 2 - RNA, 22 nt, Homo sapiens
caagcucgcuucuaugggucug

SEQ ID NO: 3 - RNA, 22 nt, Homo sapiens
aacccguagauccgaacuugug

SEQ ID NO: 4 - RNA, 22 nt, Homo sapiens
caagcuuguaucuauagguaug

SEQ ID NO: 5 - RNA, 22 nt, Homo sapiens
ucccugagacccuaacuuguga

SEQ ID NO: 6 - RNA, 22 nt, Homo sapiens
acggguuaggcucuugggagcu

SEQ ID NO: 7 - RNA, 22 nt, Homo sapiens
ucccugagacccuaacuuguga

SEQ ID NO: 8 - RNA, 22 nt, Homo sapiens
ucacaagucaggcucuugggac

SEQ ID NO: 9 - RNA, 22 nt, Homo sapiens
caucuuccaguacaguguugga

SEQ ID NO: 10 - RNA, 22 nt, Homo sapiens
uaacacugucugguaaagaugg

SEQ ID NO: 11 - RNA, 21 nt, Homo sapiens
cugaccuaugaauugacagcc

SEQ ID NO: 12 - RNA, 22 nt, Homo sapiens
cugccaauuccauaggucacag

SEQ ID NO: 13 - RNA, 22 nt, Homo sapiens
uguaacagcaacuccaugugga

SEQ ID NO: 14 - RNA, 22 nt, Homo sapiens
ccaguggggcugcuguuaucug

SEQ ID NO: 15 - RNA, 22 nt, Homo sapiens
caucuuaccggacagugcugga

SEQ ID NO: 16 - RNA, 22 nt, Homo sapiens
uaacacugucugguaacgaugu

SEQ ID NO: 17 - RNA, 22 nt, Homo sapiens
caucuuacugggcagcauugga

SEQ ID NO: 18 - RNA, 22 nt, Homo sapiens
uaauacugccugguaaugauga

SEQ ID NO: 19 - RNA, 21 nt, Homo sapiens
uugugcuugaucuaaccaugu

SEQ ID NO: 20 - RNA, 22 nt, Homo sapiens
augguuccgucaagcaccaugg

SEQ ID NO: 21 - RNA, 21 nt, Homo sapiens
uugugcuugaucuaaccaugu

SEQ ID NO: 22 - RNA, 22 nt, Homo sapiens
caugguucugucaagcaccgcg

SEQ ID NO: 23 - RNA, 23 nt, Homo sapiens
aaugacacgaucacucccguuga

SEQ ID NO: 24 - RNA, 22 nt, Homo sapiens
aucgggaaugucguguccgccc

SEQ ID NO: 25 - RNA, 22 nt, Homo sapiens
uaauacugucugguaaaaccgu

SEQ ID NO: 26 - RNA, 22 nt, Homo sapiens
caugccuugaguguaggaccgu

SEQ ID NO: 27 - RNA, 22 nt, Homo sapiens
ccucccacacccaaggcuugca

SEQ ID NO: 28 - RNA, 22 nt, Homo sapiens
ugagguaguagguuguaugguu

SEQ ID NO: 29 - RNA, 22 nt, Homo sapiens
cuguacaaccuucuagcuuucc

SEQ ID NO: 30 - DNA, 15 bp, Artificial Sequence
  Completely synthesized in the lab.
aacccgtagatccga

SEQ ID NO: 31 - DNA, 15 bp, Artificial Sequence
  Completely synthesized in the lab.
acccgtagatccgat

SEQ ID NO: 32 - DNA, 15 bp, Artificial Sequence
  Completely synthesized in the lab.
cccgtagatccgatc

SEQ ID NO: 33 - DNA, 15 bp, Artificial Sequence
  Completely synthesized in the lab.
ccgtagatccgatct

SEQ ID NO: 34 - DNA, 15 bp, Artificial Sequence
  Completely synthesized in the lab.
cgtagatccgatctt

SEQ ID NO: 35 - DNA, 15 bp, Artificial Sequence
  Completely synthesized in the lab.
gtagatccgatcttg

SEQ ID NO: 36 - DNA, 15 bp, Artificial Sequence
  Completely synthesized in the lab.
tagatccgatcttgt

SEQ ID NO: 37 - DNA, 15 bp, Artificial Sequence
  Completely synthesized in the lab.
aacccgtagatccga

SEQ ID NO: 38 - DNA, 15 bp, Artificial Sequence
  Completely synthesized in the lab.
acccgtagatccgaa

SEQ ID NO: 39 - DNA, 15 bp, Artificial Sequence
  Completely synthesized in the lab.
cccgtagatccgaac

SEQ ID NO: 40 - DNA, 15 bp, Artificial Sequence
  Completely synthesized in the lab.
ccgtagatccgaact

SEQ ID NO: 41 - DNA, 15 bp, Artificial Sequence
  Completely synthesized in the lab.
cgtagatccgaactt

SEQ ID NO: 42 - DNA, 15 bp, Artificial Sequence
  Completely synthesized in the lab.
gtagatccgaacttg

SEQ ID NO: 43 - DNA, 15 bp, Artificial Sequence
  Completely synthesized in the lab.
tagatccgaacttgt

SEQ ID NO: 44 - DNA, 15 bp, Artificial Sequence
  Completely synthesized in the lab.
tccctgagaccctaa

SEQ ID NO: 45 - DNA, 15 bp, Artificial Sequence
  Completely synthesized in the lab.
ccctgagaccctaac

SEQ ID NO: 46 - DNA, 15 bp, Artificial Sequence
  Completely synthesized in the lab.
cctgagaccctaact

SEQ ID NO: 47 - DNA, 15 bp, Artificial Sequence
  Completely synthesized in the lab.
ctgagaccctaactt

SEQ ID NO: 48 - DNA, 15 bp, Artificial Sequence
  Completely synthesized in the lab1.
tgagaccctaacttg

SEQ ID NO: 49 - DNA, 15 bp, Artificial Sequence
  Completely synthesized in the lab.
gagaccctaacttgt

SEQ ID NO: 50 - DNA, 15 bp, Artificial Sequence
  Completely synthesized in the lab.
agaccctaacttgtg

SEQ ID NO: 51 - DNA, 15 bp, Artificial Sequence
  Completely synthesized in the lab.
tccctgagaccctaa

SEQ ID NO: 52 - DNA, 15 bp, Artificial Sequence
  Completely synthesized in the lab.
ccctgagaccctaac

SEQ ID NO: 53 - DNA, 15 bp, Artificial Sequence
  Completely synthesized in the lab.
cctgagaccctaact

SEQ ID NO: 54 - DNA, 15 bp, Artificial Sequence
  Completely synthesized in the lab.
ctgagaccctaactt

SEQ ID NO: 55 - DNA, 15 bp, Artificial Sequence
  Completely synthesized in the lab.
tgagaccctaacttg

SEQ ID NO: 56 - DNA, 15 bp, Artificial Sequence
  Completely synthesized in the lab.
gagaccctaacttgt

SEQ ID NO: 57 - DNA, 15 bp, Artificial Sequence
  Completely synthesized in the lab.
agaccctaacttgtg

SEQ ID NO: 58 - DNA, 15 bp, Artificial Sequence
  Completely synthesized in the lab.
catcttccagtacag

SEQ ID NO: 59 - DNA, 15 bp, Artificial Sequence
  Completely synthesized in the lab.
atcttccagtacagt

SEQ ID NO: 60 - DNA, 15 bp, Artificial Sequence
  Completely synthesized in the lab.
tcttccagtacagtg

SEQ ID NO: 61 - DNA, 15 bp, Artificial Sequence
  Completely synthesized in the lab.
cttccagtacagtgt

SEQ ID NO: 62 - DNA, 15 bp, Artificial Sequence
  Completely synthesized in the lab.
ttccagtacagtgtt

SEQ ID NO: 63 - DNA, 15 bp, Artificial Sequence
  Completely synthesized in the lab.
tccagtacagtgttg

SEQ ID NO: 64 - DNA, 15 bp, Artificial Sequence
  Completely synthesized in the lab.
ccagtacagtgttgg

SEQ ID NO: 65 - DNA, 15 bp, Artificial Sequence
  Completely synthesized in the lab.
ctgacctatgaattg

SEQ ID NO: 66 - DNA, 15 bp, Artificial Sequence
  Completely synthesized in the lab.
tgacctatgaattga

SEQ ID NO: 67 - DNA, 15 bp, Artificial Sequence
  Completely synthesized in the lab.
gacctatgaattgac

SEQ ID NO: 68 - DNA, 15 bp, Artificial Sequence
  Completely synthesized in the lab.
acctatgaattgaca

SEQ ID NO: 69 - DNA, 15 bp, Artificial Sequence
  Completely synthesized in the lab.
cctatgaattgacag

SEQ ID NO: 70 - DNA, 15 bp, Artificial Sequence
  Completely synthesized in the lab.
ctatgaattgacagc

SEQ ID NO: 71 - DNA, 15 bp, Artificial Sequence
  Completely synthesized in the lab.
tatgaattgacagcc

SEQ ID NO: 72 - DNA, 15 bp, Artificial Sequence
  Completely synthesized in the lab.
tgtaacagcaactcc

SEQ ID NO: 73 - DNA, 15 bp, Artificial Sequence
  Completely synthesized in the lab.
gtaacagcaactcca

SEQ ID NO: 74 - DNA, 15 bp, Artificial Sequence
  Completely synthesized in the lab.
taacagcaactccat

SEQ ID NO: 75 - DNA, 15 bp, Artificial Sequence
  Completely synthesized in the lab.
aacagcaactccatg

SEQ ID NO: 76 - DNA, 15 bp, Artificial Sequence
  Completely synthesized in the lab.
acagcaactccatgt

SEQ ID NO: 77 - DNA, 15 bp, Artificial Sequence
  Completely synthesized in the lab.
cagcaactccatgtg

SEQ ID NO: 78 - DNA, 15 bp, Artificial Sequence
  Completely synthesized in the lab.
agcaactccatgtgg

SEQ ID NO: 79 - DNA, 15 bp, Artificial Sequence
  Completely synthesized in the lab.
catcttaccggacag

SEQ ID NO: 80 - DNA, 15 bp, Artificial Sequence
  Completely synthesized in the lab.
atcttaccggacagt

SEQ ID NO: 81 - DNA, 15 bp, Artificial Sequence
  Completely synthesized in the lab.
tcttaccggacagtg

SEQ ID NO: 82 - DNA, 15 bp, Artificial Sequence
  Completely synthesized in the lab.
cttaccggacagtgc

SEQ ID NO: 83 - DNA, 15 bp, Artificial Sequence
  Completely synthesized in the lab.
ttaccggacagtgct

SEQ ID NO: 84 - DNA, 15 bp, Artificial Sequence
  Completely synthesized in the lab.
taccggacagtgctg

SEQ ID NO: 85 - DNA, 14 bp, Artificial Sequence
  Completely synthesized in the lab.
accggacagtgctg

SEQ ID NO: 86 - DNA, 15 bp, Artificial Sequence
  Completely synthesized in the lab.
catcttactgggcag

SEQ ID NO: 87 - DNA, 15 bp, Artificial Sequence
  Completely synthesized in the lab.
atcttactgggcagc

SEQ ID NO: 88 - DNA, 15 bp, Artificial Sequence
  Completely synthesized in the lab.
tcttactgggcagca

SEQ ID NO: 89 - DNA, 15 bp, Artificial Sequence
  Completely synthesized in the lab.
cttactgggcagcat

SEQ ID NO: 90 - DNA, 15 bp, Artificial Sequence
  Completely synthesized in the lab.
ttactgggcagcatt

SEQ ID NO: 91 - DNA, 15 bp, Artificial Sequence
  Completely synthesized in the lab.
tactgggcagcattg

SEQ ID NO: 92 - DNA, 15 bp, Artificial Sequence
  Completely synthesized in the lab.
actgggcagcattgg

SEQ ID NO: 93 - DNA, 15 bp, Artificial Sequence
  Completely synthesized in the lab.
ttgtgcttgatctaa

SEQ ID NO: 94 - DNA, 15 bp, Artificial Sequence
  Completely synthesized in the lab.
tgtgcttgatctaac

SEQ ID NO: 95 - DNA, 15 bp, Artificial Sequence
  Completely synthesized in the lab.
gtgcttgatctaacc

SEQ ID NO: 96 - DNA, 15 bp, Artificial Sequence
  Completely synthesized in the lab.
tgcttgatctaacca

SEQ ID NO: 97 - DNA, 15 bp, Artificial Sequence
  Completely synthesized in the lab.
gcttgatctaaccat

SEQ ID NO: 98 - DNA, 15 bp, Artificial Sequence
  Completely synthesized in the lab.
cttgatctaaccatg

SEQ ID NO: 99 - DNA, 15 bp, Artificial Sequence
  Completely synthesized in the lab.
ttgatctaaccatgt

SEQ ID NO: 100 - DNA, 15 bp, Artificial Sequence
  Completely synthesized in the lab.
ttgtgcttgatctaa

SEQ ID NO: 101 - DNA, 15 bp, Artificial Sequence
  Completely synthesized in the lab.
tgtgcttgatctaac

SEQ ID NO: 102 - DNA, 15 bp, Artificial Sequence
  Completely synthesized in the lab.
gtgcttgatctaacc

SEQ ID NO: 103 - DNA, 15 bp, Artificial Sequence
  Completely synthesized in the lab.
tgcttgatctaacca

SEQ ID NO: 104 - DNA, 15 bp, Artificial Sequence
  Completely synthesized in the lab.
gcttgatctaaccat

SEQ ID NO: 105 - DNA, 15 bp, Artificial Sequence
  Completely synthesized in the lab.
cttgatctaaccatg

SEQ ID NO: 106 - DNA, 15 bp, Artificial Sequence
  Completely synthesized in the lab.
ttgatctaaccatgt

SEQ ID NO: 107 - DNA, 15 bp, Artificial Sequence
  Completely synthesized in the lab.
aatgacacgatcact

SEQ ID NO: 108 - DNA, 15 bp, Artificial Sequence
  Completely synthesized in the lab.
atgacacgatcactc

SEQ ID NO: 109 - DNA, 15 bp, Artificial Sequence
  Completely synthesized in the lab.
tgacacgatcactcc

SEQ ID NO: 110 - DNA, 15 bp, Artificial Sequence
  Completely synthesized in the lab.
gacacgatcactccc

SEQ ID NO: 111 - DNA, 15 bp, Artificial Sequence
  Completely synthesized in the lab.
acacgatcactcccg

SEQ ID NO: 112 - DNA, 15 bp, Artificial Sequence
  Completely synthesized in the lab.
cacgatcactcccgt

SEQ ID NO: 113 - DNA, 15 bp, Artificial Sequence
  Completely synthesized in the lab.
acgatcactcccgtt

SEQ ID NO: 114 - DNA, 15 bp, Artificial Sequence
  Completely synthesized in the lab.
catgccttgagtgta

SEQ ID NO: 115 - DNA, 15 bp, Artificial Sequence
  Completely synthesized in the lab.
atgccttgagtgtag

SEQ ID NO: 116 - DNA, 15 bp, Artificial Sequence
  Completely synthesized in the lab.
tgccttgagtgtagg

SEQ ID NO: 117 - DNA, 15 bp, Artificial Sequence
  Completely synthesized in the lab.
gccttgagtgtagga

SEQ ID NO: 118 - DNA, 15 bp, Artificial Sequence
  Completely synthesized in the lab.
ccttgagtgtaggac

SEQ ID NO: 119 - DNA, 15 bp, Artificial Sequence
  Completely synthesized in the lab.
cttgagtgtaggacc

SEQ ID NO: 120 - DNA, 15 bp, Artificial Sequence
  Completely synthesized in the lab.
ttgagtgtaggaccg

SEQ ID NO: 121 - DNA, 15 bp, Artificial Sequence
  Completely synthesized in the lab.
tgaggtagtaggttg

SEQ ID NO: 122 - DNA, 15 bp, Artificial Sequence
  Completely synthesized in the lab.
gaggtagtaggttgt

SEQ ID NO: 123 - DNA, 15 bp, Artificial Sequence
  Completely synthesized in the lab.
aggtagtaggttgta

SEQ ID NO: 124 - DNA, 15 bp, Artificial Sequence
  Completely synthesized in the lab.
ggtagtaggttgtat

SEQ ID NO: 125 - DNA, 15 bp, Artificial Sequence
  Completely synthesized in the lab.
gtagtaggttgtatg

SEQ ID NO: 126 - DNA, 15 bp, Artificial Sequence
  Completely synthesized in the lab.
tagtaggttgtatgg

SEQ ID NO: 127 - DNA, 15 bp, Artificial Sequence
  Completely synthesized in the lab.
agtaggttgtatggt

SEQ ID NO: 128 - DNA, 15 bp, Artificial Sequence
  Completely synthesized in the lab.
caagctcgcttctat

SEQ ID NO: 129 - DNA, 15 bp, Artificial Sequence
  Completely synthesized in the lab.
aagctcgcttctatg

SEQ ID NO: 130 - DNA, 15 bp, Artificial Sequence
  Completely synthesized in the lab.
agctcgcttctatgg

SEQ ID NO: 131 - DNA, 15 bp, Artificial Sequence
  Completely synthesized in the lab.
gctcgcttctatggg

SEQ ID NO: 132 - DNA, 15 bp, Artificial Sequence
  Completely synthesized in the lab.
ctcgcttctatgggt

SEQ ID NO: 133 - DNA, 15 bp, Artificial Sequence
  Completely synthesized in the lab.
tcgcttctatgggtc

SEQ ID NO: 134 - DNA, 15 bp, Artificial Sequence
  Completely synthesized in the lab.
cgcttctatgggtct

SEQ ID NO: 135 - DNA, 15 bp, Artificial Sequence
  Completely synthesized in the lab.
caagcttgtatctat

SEQ ID NO: 136 - DNA, 15 bp, Artificial Sequence
  Completely synthesized in the lab.
aagcttgtatctata

SEQ ID NO: 137 - DNA, 15 bp, Artificial Sequence
  Completely synthesized in the lab.
agcttgtatctatag

SEQ ID NO: 138 - DNA, 15 bp, Artificial Sequence
  Completely synthesized in the lab.
gcttgtatctatagg

SEQ ID NO: 139 - DNA, 15 bp, Artificial Sequence
  Completely synthesized in the lab.
cttgtatctataggt

SEQ ID NO: 140 - DNA, 15 bp, Artificial Sequence
  Completely synthesized in the lab.
ttgtatctataggta

SEQ ID NO: 141 - DNA, 15 bp, Artificial Sequence
  Completely synthesized in the lab.
tgtatctataggtat

SEQ ID NO: 142 - DNA, 15 bp, Artificial Sequence
  Completely synthesized in the lab.
acgggttaggctctt

SEQ ID NO: 143 - DNA, 15 bp, Artificial Sequence
  Completely synthesized in the lab.
cgggttaggctcttg

SEQ ID NO: 144 - DNA, 15 bp, Artificial Sequence
  Completely synthesized in the lab.
gggttaggctcttgg

SEQ ID NO: 145 - DNA, 15 bp, Artificial Sequence
  Completely synthesized in the lab.
ggttaggctcttggg

SEQ ID NO: 146 - DNA, 15 bp, Artificial Sequence
  Completely synthesized in the lab.
gttaggctcttggga

SEQ ID NO: 147 - DNA, 15 bp, Artificial Sequence
  Completely synthesized in the lab.
ttaggctcttgggag

SEQ ID NO: 148 - DNA, 15 bp, Artificial Sequence
  Completely synthesized in the lab.
taggctcttgggagc

SEQ ID NO: 149 - DNA, 15 bp, Artificial Sequence
  Completely synthesized in the lab.
tcacaagtcaggctc

SEQ ID NO: 150 - DNA, 15 bp, Artificial Sequence
  Completely synthesized in the lab.
cacaagtcaggctct

SEQ ID NO: 151 - DNA, 15 bp, Artificial Sequence
  Completely synthesized in the lab.
acaagtcaggctctt

SEQ ID NO: 152 - DNA, 15 bp, Artificial Sequence
  Completely synthesized in the lab.
caagtcaggctcttg

SEQ ID NO: 153 - DNA, 15 bp, Artificial Sequence
  Completely synthesized in the lab.
aagtcaggctcttgg

SEQ ID NO: 154 - DNA, 15 bp, Artificial Sequence
  Completely synthesized in the lab.
agtcaggctcttggg

SEQ ID NO: 155 - DNA, 15 bp, Artificial Sequence
  Completely synthesized in the lab.
gtcaggctcttggga

SEQ ID NO: 156 - DNA, 15 bp, Artificial Sequence
  Completely synthesized in the lab.
taacactgtctggta

SEQ ID NO: 157 - DNA, 15 bp, Artificial Sequence
  Completely synthesized in the lab.
aacactgtctggtaa

SEQ ID NO: 158 - DNA, 15 bp, Artificial Sequence
  Completely synthesized in the lab.
acactgtctggtaaa

SEQ ID NO: 159 - DNA, 15 bp, Artificial Sequence
  Completely synthesized in the lab.
cactgtctggtaaag

SEQ ID NO: 160 - DNA, 15 bp, Artificial Sequence
  Completely synthesized in the lab.
actgtctggtaaaga

SEQ ID NO: 161 - DNA, 15 bp, Artificial Sequence
  Completely synthesized in the lab.
ctgtctggtaaagat

SEQ ID NO: 162 - DNA, 15 bp, Artificial Sequence
  Completely synthesized in the lab.
tgtctggtaaagatg

SEQ ID NO: 163 - DNA, 15 bp, Artificial Sequence
  Completely synthesized in the lab.
ctgccaattccatag

SEQ ID NO: 164 - DNA, 15 bp, Artificial Sequence
  Completely synthesized in the lab.
tgccaattccatagg

SEQ ID NO: 165 - DNA, 15 bp, Artificial Sequence
  Completely synthesized in the lab.
gccaattccataggt

SEQ ID NO: 166 - DNA, 15 bp, Artificial Sequence
  Completely synthesized in the lab.
ccaattccataggtc

SEQ ID NO: 167 - DNA, 15 bp, Artificial Sequence
  Completely synthesized in the lab.
caattccataggtca

SEQ ID NO: 168 - DNA, 15 bp, Artificial Sequence
  Completely synthesized in the lab.
aattccataggtcac

SEQ ID NO: 169 - DNA, 15 bp, Artificial Sequence
  Completely synthesized in the lab.
attccataggtcaca

SEQ ID NO: 170 - DNA, 15 bp, Artificial Sequence
  Completely synthesized in the lab.
ccagtggggctgctg

SEQ ID NO: 171 - DNA, 15 bp, Artificial Sequence
  Completely synthesized in the lab.
cagtggggctgctgt

SEQ ID NO: 172 - DNA, 15 bp, Artificial Sequence
  Completely synthesized in the lab.
agtggggctgctgtt

SEQ ID NO: 173 - DNA, 15 bp, Artificial Sequence
  Completely synthesized in the lab.
gtggggctgctgtta

SEQ ID NO: 174 - DNA, 15 bp, Artificial Sequence
  Completely synthesized in the lab.
tggggctgctgttat

SEQ ID NO: 175 - DNA, 15 bp, Artificial Sequence
  Completely synthesized in the lab.
ggggctgctgttatc

SEQ ID NO: 176 - DNA, 15 bp, Artificial Sequence
  Completely synthesized in the lab.
gggctgctgttatct

SEQ ID NO: 177 - DNA, 15 bp, Artificial Sequence
  Completely synthesized in the lab.
taacactgtctggta

SEQ ID NO: 178 - DNA, 15 bp, Artificial Sequence
  Completely synthesized in the lab.
aacactgtctggtaa

SEQ ID NO: 179 - DNA, 15 bp, Artificial Sequence
  Completely synthesized in the lab.
acactgtctggtaac

SEQ ID NO: 180 - DNA, 15 bp, Artificial Sequence
  Completely synthesized in the lab.
cactgtctggtaacg

SEQ ID NO: 181 - DNA, 15 bp, Artificial Sequence
  Completely synthesized in the lab.
actgtctggtaacga

SEQ ID NO: 182 - DNA, 15 bp, Artificial Sequence
  Completely synthesized in the lab.
ctgtctggtaacgat

SEQ ID NO: 183 - DNA, 15 bp, Artificial Sequence
  Completely synthesized in the lab.
tgtctggtaacgatg

SEQ ID NO: 184 - DNA, 15 bp, Artificial Sequence
  Completely synthesized in the lab.
taatactgcctggta

SEQ ID NO: 185 - DNA, 15 bp, Artificial Sequence
  Completely synthesized in the lab.
aatactgcctggtaa

SEQ ID NO: 186 - DNA, 15 bp, Artificial Sequence
  Completely synthesized in the lab.
atactgcctggtaat

SEQ ID NO: 187 - DNA, 15 bp, Artificial Sequence
  Completely synthesized in the lab.
tactgcctggtaatg

SEQ ID NO: 188 - DNA, 15 bp, Artificial Sequence
  Completely synthesized in the lab.
actgcctggtaatga

SEQ ID NO: 189 - DNA, 15 bp, Artificial Sequence
  Completely synthesized in the lab.
ctgcctggtaatgat

SEQ ID NO: 190 - DNA, 15 bp, Artificial Sequence
  Completely synthesized in the lab.
tgcctggtaatgatg

SEQ ID NO: 191 - DNA, 15 bp, Artificial Sequence
  Completely synthesized in the lab.
atggttccgtcaagc

SEQ ID NO: 192 - DNA, 15 bp, Artificial Sequence
  Completely synthesized in the lab.
tggttccgtcaagca

SEQ ID NO: 193 - DNA, 15 bp, Artificial Sequence
  Completely synthesized in the lab.
ggttccgtcaagcac

SEQ ID NO: 194 - DNA, 15 bp, Artificial Sequence
  Completely synthesized in the lab.
gttccgtcaagcacc

SEQ ID NO: 195 - DNA, 15 bp, Artificial Sequence
  Completely synthesized in the lab.
ttccgtcaagcacca

SEQ ID NO: 196 - DNA, 15 bp, Artificial Sequence
  Completely synthesized in the lab.
tccgtcaagcaccat

SEQ ID NO: 197 - DNA, 15 bp, Artificial Sequence
  Completely synthesized in the lab.
ccgtcaagcaccatg

SEQ ID NO: 198 - DNA, 15 bp, Artificial Sequence
  Completely synthesized in the lab.
catggttctgtcaag

SEQ ID NO: 199 - DNA, 15 bp, Artificial Sequence
  Completely synthesized in the lab.
atggttctgtcaagc

SEQ ID NO: 200 - DNA, 15 bp, Artificial Sequence
  Completely synthesized in the lab.
tggttctgtcaagca

SEQ ID NO: 201 - DNA, 15 bp, Artificial Sequence
  Completely synthesized in the lab.
ggttctgtcaagcac

SEQ ID NO: 202 - DNA, 15 bp, Artificial Sequence
  Completely synthesized in the lab.
gttctgtcaagcacc

SEQ ID NO: 203 - DNA, 15 bp, Artificial Sequence
  Completely synthesized in the lab.
ttctgtcaagcaccg

SEQ ID NO: 204 - DNA, 15 bp, Artificial Sequence
  Completely synthesized in the lab.
tctgtcaagcaccgc

SEQ ID NO: 205 - DNA, 15 bp, Artificial Sequence
  Completely synthesized in the lab.
atcgggaatgtcgtg

SEQ ID NO: 206 - DNA, 15 bp, Artificial Sequence
  Completely synthesized in the lab.
tcgggaatgtcgtgt

SEQ ID NO: 207 - DNA, 15 bp, Artificial Sequence
  Completely synthesized in the lab.
cgggaatgtcgtgtc

SEQ ID NO: 208 - DNA, 15 bp, Artificial Sequence
  Completely synthesized in the lab.
gggaatgtcgtgtcc

SEQ ID NO: 209 - DNA, 15 bp, Artificial Sequence
  Completely synthesized in the lab.
ggaatgtcgtgtccg

SEQ ID NO: 210 - DNA, 15 bp, Artificial Sequence
  Completely synthesized in the lab.
gaatgtcgtgtccgc

SEQ ID NO: 211 - DNA, 15 bp, Artificial Sequence
  Completely synthesized in the lab.
aatgtcgtgtccgcc

SEQ ID NO: 212 - DNA, 15 bp, Artificial Sequence
  Completely synthesized in the lab.
taatactgtctggta

SEQ ID NO: 213 - DNA, 15 bp, Artificial Sequence
  Completely synthesized in the lab.
aatactgtctggtaa

SEQ ID NO: 214 - DNA, 15 bp, Artificial Sequence
  Completely synthesized in the lab.
atactgtctggtaaa

SEQ ID NO: 215 - DNA, 15 bp, Artificial Sequence
  Completely synthesized in the lab.
tactgtctggtaaaa

SEQ ID NO: 216 - DNA, 15 bp, Artificial Sequence
  Completely synthesized in the lab.
actgtctggtaaaac

SEQ ID NO: 217 - DNA, 15 bp, Artificial Sequence
  Completely synthesized in the lab.
ctgtctggtaaaacc

SEQ ID NO: 218 - DNA, 15 bp, Artificial Sequence
  Completely synthesized in the lab.
tgtctggtaaaaccg

SEQ ID NO: 219 - DNA, 15 bp, Artificial Sequence
  Completely synthesized in the lab.
cctcccacacccaag

SEQ ID NO: 220 - DNA, 15 bp, Artificial Sequence
  Completely synthesized in the lab.
ctcccacacccaagg

SEQ ID NO: 221 - DNA, 15 bp, Artificial Sequence
  Completely synthesized in the lab.
tcccacacccaaggc

SEQ ID NO: 222 - DNA, 14 bp, Artificial Sequence
  Completely synthesized in the lab.
cccacacccaaggt

SEQ ID NO: 223 - DNA, 15 bp, Artificial Sequence
  Completely synthesized in the lab.
ccacacccaaggctt

SEQ ID NO: 224 - DNA, 15 bp, Artificial Sequence
  Completely synthesized in the lab.
cacacccaaggcttg

SEQ ID NO: 225 - DNA, 15 bp, Artificial Sequence
  Completely synthesized in the lab.
acacccaaggcttgc

SEQ ID NO: 226 - DNA, 15 bp, Artificial Sequence
  Completely synthesized in the lab.
ctgtacaaccttcta

SEQ ID NO: 227 - DNA, 15 bp, Artificial Sequence
  Completely synthesized in the lab.
tgtacaaccttctag

SEQ ID NO: 228 - DNA, 15 bp, Artificial Sequence
  Completely synthesized in the lab.
gtacaaccttctagc

SEQ ID NO: 229 - DNA, 15 bp, Artificial Sequence
  Completely synthesized in the lab.
tacaaccttctagct

SEQ ID NO: 230 - DNA, 15 bp, Artificial Sequence
  Completely synthesized in the lab.
acaaccttctagctt

SEQ ID NO: 231 - DNA, 15 bp, Artificial Sequence
  Completely synthesized in the lab.
caaccttctagcttt

SEQ ID NO: 232 - DNA, 15 bp, Artificial Sequence
  Completely synthesized in the lab.
aaccttctagctttc

SEQ ID NO: 233 - DNA, 15 bp, Artificial Sequence
  Completely synthesized in the lab.
ttgggcatctaggca

SEQ ID NO: 234 - RNA, 16 nt, Artificial Sequence
  Completely synthesized in the lab.
cacaagaucggaucua